(19) United States
(12) Patent Application Publication
(10) Pub. No.: US 2025/0144040 A1
(43) Pub. Date: May 8, 2025

(54) COMPOSITE PARTICLE FORMULATIONS AND APPLICATIONS THEREOF

(71) Applicant: THE UNIVERSITY OF NORTH CAROLINA AT CHAPEL HILL, Chapel Hill, NC (US)

(72) Inventors: Alexander Kabanov, Chapel Hill, NC (US); Jacob Ramsey, Chapel Hill, NC (US)

(21) Appl. No.: 18/730,206

(22) PCT Filed: Jan. 20, 2023
(86) PCT No.: PCT/US23/11250

§ 371 Date: Jul. 18, 2024

Related U.S. Application Data
(60) Provisional application No. 63/301,251, filed on Jan. 20, 2022.

(51) Int. Cl. A61K 9/51; A61K 9/19; A61K 31/337; A61K 31/343; A61K 31/404; A61K 31/437; A61K 31/47; A61K 31/4965; A61K 31/569; A61K 31/58; A61K 31/675; A61P 31/14

(52) U.S. Cl. CPC A61K 9/5138; A61K 9/19; A61K 31/337; A61K 31/343; A61K 31/404; A61K 31/437; A61K 31/47; A61K 31/4965; A61K 31/569; A61K 31/58; A61K 31/675; A61P 31/14

(57) ABSTRACT

Composite particle including the following: (1) a nanocrystal of an organic compound, and (2) a block copolymer associated with one or more surfaces of the nanocrystal of an organic compound. The organic compound, in some embodiments, is a pharmaceutical compound, a therapeutic compound, and/or a bioactive compound. The block copolymer may be a block copolymer with at least one hydrophilic poly(2-oxazoline) block. Pharmaceutical compositions may include these composite particles. The pharmaceutical compositions may include a dispersion that has an aqueous or aqueous-based continuous phase and a dispersed phase that includes the composite particles. The dispersion may be administered to sick patients, including those diagnosed with or exhibiting symptoms of COVID-19. For example, the dispersion may be aerosolized, and a patient may inhale the aerosol.

### RELATED APPLICATION DATA

The present application claims priority pursuant to Article 8 of the Patent Cooperation Treaty to U.S. Provisional Patent Application Ser. No. 63/301,251 filed Jan. 20, 2022, which is hereby incorporated by reference in its entirety.

### FIELD

The present invention relates to composite nanocrystal formulations and, in particular, to composite nanocrystal dispersions having high pharmaceutical loadings for aerosol administration.

### BACKGROUND

With an increasing number of approved drugs, the design of novel drug molecules has become more complicated. The challenge in drug design is the concurrent demand for achieving higher therapeutic potency with proper physicochemical properties and lower toxicity. Large pharmaceutical companies employ high throughput screening (HTS) to find lead compounds, but the structure optimization that takes place after HTS often results in therapeutic failure. It is well known that the increased structural complexity of therapeutic compounds is positively correlated to drug development success rate. Therefore, the use of additional strategies, such as rational drug design, is a necessity to help simplify this complex, failure-prone process. One potential approach to simplify drug development is to take advantage of drug delivery systems. Drug delivery systems can expand the design space of pharmaceutical molecules by compensating for undesirable physicochemical properties of therapeutic agents and modulating their pharmacokinetics, biodistribution, and cellular uptake.

With the advent of COVID-19 and other highly transmissible respiratory diseases, the need for robust and efficient drug delivery systems has accelerated. Aerosol administration can be particularly effective in treating such diseases. However, providing aerosols with sufficiently high pharmaceutical concentrations remains difficult, thereby limiting aerosol delivery relative to other forms, including intravenous delivery.

The need to overcome the limitations of aerosol administration is especially high for drugs that cannot be delivered orally and are limited to intravenous delivery that must be done in a clinical setting. For example, Remdesivir cannot be administered orally due to its intrinsic liver toxicity, and is typically administered intravenously. Thus, the ability to effectively administer Remdesivir outside of a clinical setting, e.g., using aerosol delivery, is desired.

### SUMMARY

In one aspect, a composite particle is disclosed herein. The composite particle comprises the following (1) a nanocrystal of an organic compound; and (2) a block copolymer associated with one or more surfaces of the nanocrystal of an organic compound, the block copolymer comprising at least one hydrophilic poly(2-oxazoline) block. In some embodiments, the composite may have an average size of 70 to 1,000 nm. A ratio of the block copolymer to the organic compound in the composite may range from 0.1:100 to 10:100 by weight or 1:100 to 10:100 by weight. The organic compound, in some embodiments, may be present in an amount of at least 80 weight percent of the particle. In some embodiments, the organic compound is present in an amount of at least 80 weight percent, at least 90 weight percent, or at least 95 weight percent of the particle. In some embodiments, the organic compound is greater than 90-99.9 weight percent of the particle.

The organic compound, in some cases, may have a solubility less than 10 mg/mL or less than 5 mg/mL in water in a pH range from 4 and 10 at 20° C. In some cases, the organic compound may have a Log(P) in a range from −2.0 to 10.0. In some embodiments, the organic compound is a pharmaceutical agent, a therapeutic agent, or combinations thereof. Non-limiting examples of organic compounds include Remdesivir, Resiquimod, Paclitaxel, Rifampicin, Fluticasone, Budenoside, Amodiaquine, Umifenovir, and Favapiravir.

The block copolymer is not so limited and in some embodiments may be a block copolymer that exhibits at least one of a foam height (cm) of less than 3 cm and a foam half-life (hr.) of less than 3 hr. In some embodiments, the hydrophilic poly(2-oxazoline) block is of the formula:

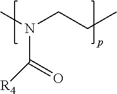

wherein R⁴ is selected from the group consisting of alkyl, cycloalkyl and alkenyl, each optionally substituted with hydroxyl, —SH, —COOR⁵, —NR⁶₂, —CONR⁷ or —CHO, wherein R⁵-R⁷ are independently selected from the group consisting of hydrogen and alkyl and wherein p is at least 5. In some embodiments, the hydrophilic poly(2-oxazoline) block comprises poly(2-methyl-2-oxazoline) and/or poly(2-ethyl-2-oxazoline).

In some embodiments, the block copolymer may further comprise a second block that is more hydrophobic than the hydrophilic poly(2-oxazoline) block. For example, the second block may include a monomer having a side chain comprising a heteroaryl moiety. The heteroaryl moiety may be a five-membered ring or six membered ring. In some embodiments, the heteroaryl moiety is of the formula:

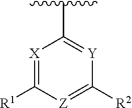

wherein X, Y and Z are heteroatoms independently selected from the group consisting of N, O, and S, and R¹ and R² are independently selected from the group consisting of hydrogen, alkyl, cycloalkyl, alkenyl, aryl, heteroaryl, amine, —SH, hydroxyl, halo, carboxyl, and —CONR³, wherein R³ is alkyl and [symbol] represents a point of attachment of the heteroaryl moiety to the monomer. In some embodiments, the second block comprises poly(2-oxazine).

In another aspect, a lyophilized powder comprising the composite particles described herein is provided. In some embodiments, the composite particles of the lyophilized powder have an average size of 70-1,000 nm. In some embodiments, the organic compound is present in an amount of at least 80 weight percent of the lyophilized powder or in an amount of at least 90 weight percent of the lyophilized powder.

In another aspect, a pharmaceutical composition comprising the composite particles described herein above is described. In addition to the composite particles, the pharmaceutical composition may also comprise one or more pharmaceutically acceptable excipients.

In some embodiments, the pharmaceutical composition may be a dispersion that comprises the following (1) an aqueous or aqueous-based continuous phase, and (2) a dispersed phase comprising the composite particles. In some embodiments, the dispersion may be produced by hydrating lyophilized powder of the composite particles. The composite particles in the dispersion may have a polydispersity index (PDI) of less than 0.3, preferably less than 0.2, yet still preferably less than 0.15. In some embodiments, the composite particles may maintain this PDI for a time period of at least 20 days. The nanocrystal of an organic compound may be present in the dispersion at a concentration of 1-50 mg/mL. The block copolymer may be present at a concentration less than 5 percent of the concentration of the nanocrystals of the organic compound. In some embodiments, the nanocrystals of the organic compound are present in an amount of at least 90 weight percent based on total weight of the composite particles, preferably of at least 95 weight percent based on total weight of the composite particles.

In another aspect, an aerosol produced by nebulizing the dispersion described herein above is described.

In another aspect, a method for treating a patient, e.g., a patient suffering from COVID-19 or another disease affecting the lungs is described. The method for treating may comprise the following steps: (1) providing a dispersion comprising an aqueous or aqueous-based continuous phase, and a dispersed phase comprising composite particles, the composite particles including a pharmaceutical compound, and block copolymer associated with one or more surfaces of the pharmaceutical compound, the block copolymer comprising at least one hydrophilic poly(2-oxazoline) block; nebulizing the dispersion into an aerosol; and administering the aerosol to the patient for inhalation. In such embodiments, the pharmaceutical compound may be present at a concentration of 1-50 mg/mL. In such embodiments, the block copolymer may be present at a concentration less than 5 percent of the concentration of the pharmaceutical compound. In some embodiments, the pharmaceutical compound is present in an amount of at least 90 weight percent based on total weight of the composite particles. In some embodiments, providing the dispersion may comprise hydrating lyophilized powder of the composite particles. In some embodiments, the pharmaceutical compound may be a compound that exhibits antiviral properties, e.g., Remdesivir.

### BRIEF DESCRIPTION OF THE DRAWINGS

FIG. 1 is a table that includes HLB values and structures for block copolymers described herein.

FIG. 2 includes an H-NMR spectrum (A), a structure (B), and block length confirmation (C) of a block copolymer described herein.

FIG. 3 is a schematic drawing showing a method for forming a stabilized nanocrystal suspension by mixing stabilizer (a block copolymer) and a pharmaceutical agent like Remdesivir (an organic compound).

FIG. 4 is a graph showing DLS intensity size distribution of nanocrystals after 10 minutes stabilized with various amounts F127 (0.0, 0.15, and 0.25 mg*mL⁻¹).

FIG. 5 is a graph showing DLS intensity size distribution of nanocrystals after 10 minutes stabilized with 0.25 mg*mL⁻¹ F127 nanocrystals redispersed after lyophilization with and without 9% sucrose (w/v).

FIG. 6 is a graph showing DLS intensity size distribution of nanocrystals after 10 minutes stabilized with 0.25 mg*mL⁻¹ P2.

FIG. 7 a graph showing DLS size by intensity distribution and PDI of nanocrystals stabilized with 0.25 mg*mL⁻¹ F127 assessed over 5 days.

FIG. 8 a graph showing DLS size by intensity distribution and PDI of nanocrystals stabilized with 0.25 mg*mL⁻¹ P2 assessed over 30 days.

FIG. 9 includes TEM images of nanocrystals.

FIG. 10 includes graphs showing aerosol output of 0.25 mg/mL F127 Remdesivir (5 mg/mL) nanocrystals: (left) the nanocrystals suspension concentration and the output rate of drug over time and (right) The MMAD, GSD, and FPF of the particles over the nebulizer output time range.

FIG. 11 includes a table showing nanocrystal stability after multiple centrifugations at various speeds, DLS Size, RDV concentration. PC1 (Post-Centrifuge 1) is the pellet suspended in water after the first centrifugation. PC2 (Post-Centrifuge 2) is the pellet suspended in water after the second centrifugation. Starting nanocrystal concentration was 4 mg/mL Remdesivir, 0.2 mg/mL P2 stabilizer.

FIG. 12 is a graph relating to methanol-free P2 stabilized Remdesivir nanocrystal suspensions. The graph shows concentration of Remdesivir (mg*mL⁻¹) and the amount of methanol (volume %) remaining in the suspension after zero, one, and two centrifugal separations and redispersion of the suspension as measured by HPLC (Remdesivir) and ¹H-NMR (methanol). Initial methanol volume % was 8.0% and after a single centrifuge was less than 0.002%

FIG. 13 is a graph relating to methanol-free P2 stabilized Remdesivir nanocrystal suspensions. The graph shows the size (diameter) and PDI of P2 nanocrystals (0.25 mg*mL⁻¹ P2, 5 mg*mL⁻¹ Remdesivir after lyophilization in 20% trehalose (w/v) and stored at ambient temperature and hydrated with ultrapure water over 1-42 days after lyophilization. Sizes shown at 0 hr. and 4 hr. after hydration.

FIG. 14 is a graph relating to methanol-free P2 stabilized Remdesivir nanocrystal suspensions. The graph shows the size (diameter) and PDI of P2 nanocrystals (0.25 mg*mL⁻¹ P2, 5%) after a single centrifugation and freeze/thaw cycle in 20% Trehalose (v/v) over 1-42 days after freezing. Sizes shown at 0 hr. and 4 hr. after thaw.

FIG. 15 is a graph relating to methanol-free P2 stabilized Remdesivir nanocrystal suspensions. It shows the intensity size distribution of P2 nanocrystals (0.25 mg*mL⁻¹ P2, 5 mg*mL⁻¹ Remdesivir) after centrifugal removal of methanol and again after a freeze/thaw cycle in 20% trehalose (w/v).

FIG. 16 is a graph relating to methanol-free P2 stabilized Remdesivir nanocrystal suspensions. It shows the size and PDI of P2 nanocrystals after freeze/thaw (20% trehalose (w/v)) (0.125-1 mg*mL⁻¹ P2) when prepared at various Remdesivir concentrations (2.5-20 mg*mL⁻¹ Remdesivir) or when concentrated after centrifugation (5 mg*mL⁻¹ to 20 mg*mL⁻¹) and then frozen or diluted (5 mg*mL⁻¹ to 2.5 mg*mL⁻¹) and then frozen.

FIG. 17 is a graph showing size (d.nm) of P2 stabilized Remdesivir nanocrystals with and without inclusion of oleic acid. Oleic Acid inclusion at 0.005 mg/mL does not change the size of Remdesivir Nanocrystals after a freeze/thaw cycle in 20% Trehalose (DLS intensity distribution) FIG. 18 is a graph showing the zeta potential of P2 (0.25 mg*mL⁻¹) nanocrystals (5 mg*mL⁻¹ Remdesivir) with and without the addition of 0.005 mg*mL⁻¹ oleic acid as additional stabilizer.

FIG. 19 is a graph that relates to in vitro analysis of drug release from P2 stabilized Remdesivir nanocrystals, and specifically, the release of the drug over time in PBS at 37° C. of nanocrystals at two concentrations and Remdesivir cyclodextrin inclusion complexes at the same concentrations.

FIG. 20 is a graph that relates to in vitro analysis of drug release from P2 stabilized Remdesivir nanocrystals, and specifically, the intensity and number distribution by DLS of nanocrystals at zero time point and after 4 hours of drug release.

FIG. 21 is a graph that relates to in vitro analysis of drug release from P2 stabilized Remdesivir nanocrystals, and specifically, the particle size corresponding to the two peaks observed in DLS intensity distribution plots at different time points during the drug release process.

FIG. 22 is a graph that relates to in vitro analysis of drug release from P2 stabilized Remdesivir nanocrystals, and specifically the percentage of particles that are in each peak in plot (FIG. 21) as measured by the number distribution on DLS.

FIG. 23 is a graph that relates to In vitro antiviral activity of the Remdesivir nanocrystals compared to Remdesivir positive control in DMSO and the P2 polymer stabilizer alone in the A549-Ace2 cell model^{[51]} (FIG. 20, FIG. 21, FIG. 22) The drug concentration in the release experiment and size measurement was either 2 mg*mL⁻¹ or 5 mg*mL⁻¹ (0.10 or 0.25 mg*mL⁻¹ P2, respectively).

FIG. 24 relates to aerosol output from nebulization of P2 stabilized nanocrystals. Particularly, it is a series of pictures showing the foaming of water only (left), P2-stabilized nanocrystals (center) and F127 stabilized nanocrystals at 0.25 mg/mL stabilizer (right).

FIG. 25 is a graphs showing viscosity values for water, 1 mg/mL Remdesivir nanocrystal concentration, and 2.5 mg/mL Remdesivir concentrations. Viscosity values are not statistically different.

FIG. 26 relates to aerosol output from nebulization of P2 stabilized nanocrystals. Particularly, it is a graph of impactor analysis showing amount of Remdesivir mass in various particle cutoff sizes for P2 stabilized nanocrystals with and without 0.0015 mg*mL⁻¹ oleic acid (initial Remdesivir concentration at 1.5 mg*mL⁻¹). Vacuum flow rate on NGI at 15 L*min⁻¹, nanocrystal suspension volume at 3 mL, 2-minute collection time. (D) MMAD, GSD, % of Mass in fine particles (FP Dose %), FP Dose (ug), and percentage of nominal Remdesivir dose which was output are calculated as described in the methods.

FIG. 27 relates to aerosol output from nebulization of P2 stabilized nanocrystals. It is a graph showing DLS particle size of P2 stabilized nanocrystals before (green-left) and after nebulization (blue-center), as well as after incubation at room temperature during the nebulization process (red-right).

FIG. 28 is a table including data relating to P2 stabilized Remdesivir with and without the addition of oleic acid.

FIG. 29 is a table including block copolymers used in some embodiments described herein.

FIG. 30A is a table characterizing block copolymers used in some embodiments described herein.

FIGS. 30B-30E provide structures of the block copolymer listed in Table 30A.

FIG. 31 is a table including foaming properties of some block copolymers used in some embodiments described herein.

FIG. 32A is a table including Active Pharmaceutical Ingredients (APIs)/organic compounds used in some embodiments described herein.

FIGS. 32B-32F provide structures of the APIs/organic compounds listed in FIG. 32A.

FIG. 33A is a table including Active Pharmaceutical Ingredients (APIs)/organic compounds used in some embodiments described herein.

FIGS. 33B-33E provide structures of the APIs/organic compounds listed in FIG. 33A.

FIG. 34 is a table reporting particle hydrodynamic diameter for Remdesivir nanocrystals, nm, based on DLS intensity spectrum.

FIG. 35 is a table reporting particle hydrodynamic diameter for Resiquimod nanocrystals, nm, based on DLS intensity spectrum.

FIG. 36 is a table reporting particle hydrodynamic diameter for Paclitaxel nanocrystals, nm, based on DLS intensity spectrum.

FIG. 37 is a table reporting particle hydrodynamic diameter for Rifampicin nanocrystals, nm, based on DLS intensity spectrum.

FIG. 38 is a table reporting particle hydrodynamic diameter for Budenoside nanocrystals, nm, based on DLS intensity spectrum.

FIG. 39 is a table reporting particle hydrodynamic diameter for Fluticasone nanocrystals, nm, based on DLS intensity spectrum.

FIG. 40 is a table reporting particle hydrodynamic diameter for Amodiaquine nanocrystals, nm, based on DLS intensity spectrum.

FIG. 41 is a table reporting particle hydrodynamic diameter for Umifenovir nanocrystals, nm, based on DLS intensity spectrum.

FIG. 42 is a table reporting particle hydrodynamic diameter for Favapiravir nanocrystals, nm, based on DLS intensity spectrum.

FIG. 43 is a table including solvent properties for solvents used to form exemplary nanoparticles described herein.

FIG. 44 is a table reporting Particle Hydrodynamic Diameter for Remdesivir, Paclitaxel, Budenoside, and Fluticasone Nanocrystals, nm, based on DLS Intensity Spectrum.

### DETAILED DESCRIPTION

### Composite Particles

In one aspect, composite particles are described herein comprising the following (1) a nanocrystal of an organic compound, and (2) block copolymer associated with one or more surfaces of the nanocrystal of an organic compound, the block copolymer comprising at least one hydrophilic poly(2-oxazoline) block. In some embodiments, the block copolymer is said to stabilize the nanocrystal of an organic compound forming a stabilized nanocrystal of an organic compound, e.g., a stabilized nanocrystal of a pharmaceutical compound. Thus, the composite particle may be a stabilized nanocrystal of an organic compound e.g., a stabilized nanocrystal of a pharmaceutical compound.

The size of the composite particle (e.g., the stabilized organic compound or the stabilized pharmaceutical compound) is not so limited. Preferably the size is in a range from about 70 nm to about 1000 nm or 70 to about 900 nm. The composite particles, for example, can have an average size of about 70 to about 800 nm, 70 to about 700 nm, 70 to about 600 nm, 70 to about 500 nm, 70 to about 400 nm, 70 to about 300 nm, 70 to about 200 nm, 70 to about 100 nm, 70 to about 90 nm, or 70 to about 80 nm. To characterize nanocrystals a variety of methods known in the art are used. The dynamic light scattering determines the particle zeta potential, the z-average size, size number, intensity or volume distribution and polydispersity over time by DLS, the particle number-average size, distribution. The nanoparticle tracking analysis determine particle size and concentration. The particle morphology is determined by the transmission electron microscopy. The drug concentration and identity present in nanocrystal suspension for example is determined by high-performance liquid chromatography (HPLC) and Matrix-assisted laser desorption/ionization-time of flight (MALDI-TOF) mass spectrometry. MALDI-TOF can also detect the presence of solvents in the formulation. The solution viscosity, and surface tension can be measured using rheometer. Solution density can be determined from values of shear and kinematic viscosity.

A ratio of the block copolymer to the organic compound may be in a range from 0.1:100 to 20:100 by weight, 1:100 to 10:100 by weight, 2:100 to 10:100 by weight, 3:100 to 10:100 by weight, 4:100 to 10:100 by weight, 5:100 to 10:100 by weight, 6:100 to 10:100 by weight, 7:100 to 10:100 by weight, 8:100 to 10:100 by weight, or 9:100 to 10:100 by weight.

As described herein, the block copolymer is associated with one more surfaces of the nanocrystal of an organic compound. In some embodiments, the nanocrystal of organic compound is present in an amount of at least 50 weight percent of the particle. For example, the nanocrystal of organic compound can present in an amount of at least 80, at least 85, at least 90, or at least 95 weight percent of the composite particle.

Block copolymer associated with one or more surfaces of the nanocrystal of an organic compound means that the block copolymer is bonded (e.g., via ionic bonding, covalent bonding, hydrogen bonding, or van der Waals forces) with one or more surfaces of nanocrystal of an organic compound.

There are two major approaches to nanocrystal preparation: top-down and bottom-up (Amanpreet Kaur, Prashantkumar Khodabhai Parmar, Sanika Jadhav, Arvind Kumar Bansal, Chapter 12—Advances in nanocrystals as drug delivery systems. In: Nanoparticle Therapeutics Production Technologies, Types of Nanoparticles, and Regulatory Aspects, 2022, Pages 413-454 https://doi.org/10.1016/B978-0-12-820757-4.00011-9). Nanoprecipitation is bottom-up technique whereas pearl/ball milling, high-pressure homogenization, microfluidizer technology, piston gap homogenization, and spray drying are top-down techniques. Examples of top-down approaches include wet-milling processes, which are commercially scalable (see for example B. Van Eerdenbrugh, G. Van Den Mooter, P. Augustijns, Int. J. Pharm. 2008, 364, 64; B. Sinha, R. H. Müller, J. P. Moschwitzer, Int. J. Pharm. 2013, 453, 126). Each of these may be used to form the composite particles described herein. A bottom-up approach is described in further detail herein below.

With regard to the methods described above, the parameters to be varied when forming the composite particles described herein may include organic compound (e.g., pharmaceutical compound or drug) concentration, stabilizer (e.g., block copolymer) concentration, stabilizer (e.g., block copolymer) structure and length, removal or replacement of organic solvent, and temperature used during the nanocrystal formation process. Suitable organic solvents, in some embodiments, include DMSO and alcohols, such as methanol and/or ethanol.

Temperature affects interactions, e.g., hydrophobic interactions, between block copolymers and the surface of the organic compound (e.g., pharmaceutical compound).

### Nanocrystal of Organic Compound

The organic compound is not so limited so long as a block copolymer as described herein may be associated with one or more surfaces thereof.

In some preferred embodiments, the organic compound may be a pharmaceutical agent, a bioactive agent, or a therapeutic agent.

In some preferred embodiments, the organic compound is hydrophobic. Hydrophobic may mean that the solubility is less than 0.1 mg/mL, less than 1 mg/mL, less 5 mg/mL, or less than 10 mg/mL in water or aqueous media in a pH range of 0-14, preferably between pH 4 and 10, particularly at 20° C. In some embodiments, the organic compound has solubility of 0.001-5 mg/mL, 0.001-1 mg/mL, 0.01-5 mg/mL, or 0.01-1 mg/mL in water. It is preferred that the solubility in water, e.g. ion-exchanged water, at 20° C., is less than 1 mg/mL, preferably less than 0.1 mg/mL or even less than 0.01 mg/mL, and in particular with a solubility of less than 0.001 mg/mL Preferably, this limited solubility is shown in water over the pH range of 4 to 10. Examples of organic compounds (e.g., active pharmaceutical ingredients or APIs) with the required solubility are Remdesivir (0.030 mg/mL), Resiquimod (0.100 mg/mL), Paclitaxel (0.005 mg/mL), Rifampicin (1.3 mg/mL), Fluticasone (0.102 mg/mL), Budenoside (0.107 mg/mL), Amodiaquine (0.0249 mg/mL), Umifenovir (0.00678 mg/mL), and Favapiravir (5 mg/mL).

In some preferred embodiments, the organic compound may have a Log(P) value less than 10, less than 9, less than 8, less than 7, less than 6, less than 5, less than 4, less than 3, less than 2, or less than 1. In some preferred embodiments, a Log(P) value may be from −2.0 to 10.0, or from −1.0 to 5.0. The log P value, as commonly known, is the logarithm of the partition coefficient observed for a species A between water and n-octanol. In particular, the partition coefficient P of a species A is defined as the ratio P=[A]_{n-octanol}/[A]_{water}, wherein [A] indicates the concentration of A in the respective phase. The more hydrophilic substance will have higher concentrations in water. Typically, the volumes of water and octanol are the same for the measurement. Examples of organic compounds (e.g., active pharmaceutical ingredients or APIs) with the required Log(P) are Remdesivir (1.9), Resiquimod (1.3), Paclitaxel (2.5), Rifampicin (4.9), Fluticasone (3.2), Budenoside (2.5), Amodiaquine (2.6), Umifenovir (4.4), and Favapiravir (−0.6).

Other examples of suitable organic compounds include, but are not limited to, testosterone, testosterone enanthate, testosterone cypionate, methyltestosterone, amphotericin B, nifedipine, griseofulvin, taxanes (including, without limitation, paclitaxel, docetaxel, larotaxel, ortataxel, tesetaxel and the like), doxorubicin, daunomycin, indomethacin, ibuprofen, etoposide, cyclosporin A, vitamin E, nifedipine, griseofulvin, a taxane, amphotericin B, etoposide or cyclosporin A.

### Block Copolymer Comprising at Least One Hydrophilic Poly(2-Oxazoline) Block

The block polymer associated with one or more surfaces of a nanocrystal of the organic compound is not so limited, but is preferably a biocompatible block copolymer.

In some preferred embodiments, the block copolymer used may exhibit anti-foaming properties. For example, a foam height (cm) less than 3 cm is preferred, preferably less than 2.5 cm, and most preferably less than 2 cm. A foam half-life (hr.) less than 4 hr. is preferable, preferably less than 3.0 hr., more preferably less than 2.5 hr., even more preferably less than 2 or 1.5 hr., and most preferably less than about 1 hr. In some embodiments, the copolymer preferably has a foam half-life less than 3 hr. and a foam height less than 3 cm. The specific procedure to measure foaming is described below in Inventive Examples 3-11.

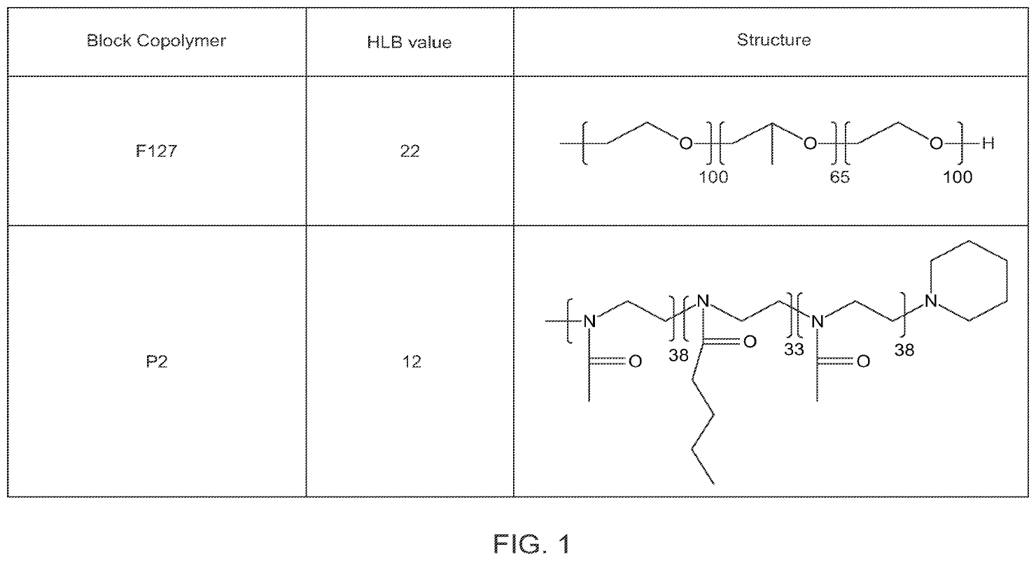

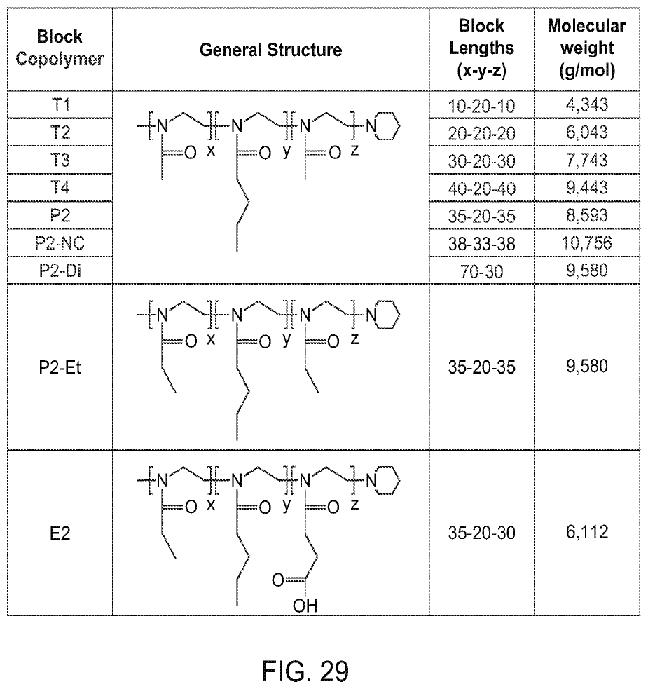

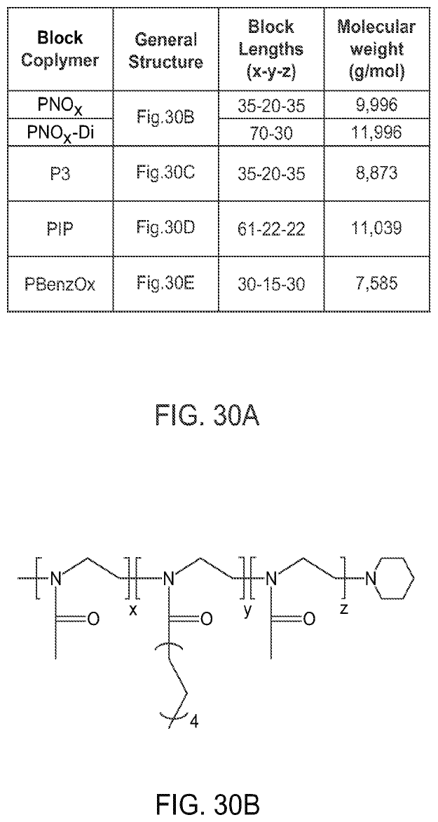

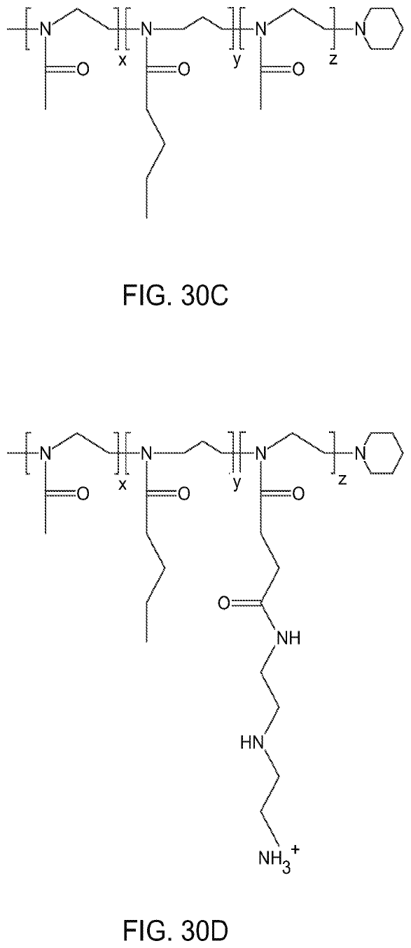

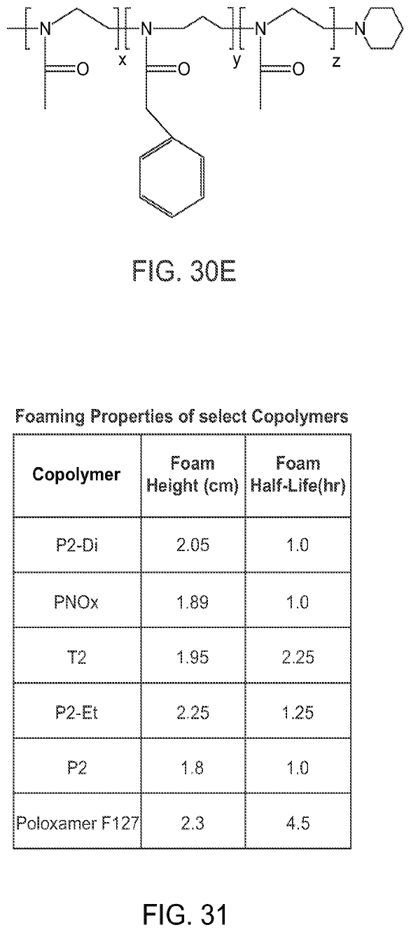

Some specific, but not limiting, examples of block copolymers as described herein are found in the tables in FIG. 1, FIG. 29, and FIGS. 30B-D.

In a particular embodiment, the biocompatible, block copolymer associated with one or more surfaces of the organic compound comprises at least one hydrophilic block A and at least one hydrophobic block B. The at least one hydrophilic block A and at least one hydrophobic block B can be attached through linkages which are stable or labile (e.g., biodegradable under physiological conditions (e.g., by the action of biologically formed entities which can be enzymes or other products of the organism)). The preferred block copolymer is either diblock or triblock copolymer having A-B, B-A, A-B-A′, A-A′-B, B-A-A′, B-A-B′, B—B′-A, or A-B—B′, where A and A′ independently of each other are same or different hydrophilic blocks, and B and B′ independently of each other are same or hydrophobic blocks. Although the hydrophilic block of the polymer preferably comprises at least one poly(2-oxazoline), the hydrophilic block may also comprise at least one polyethyleneoxide, polyester, or polyamino acid (e.g., poly(glutamic acid) or poly(aspartic acid)) or block thereof. In some embodiments, the hydrophilic block may comprise one or more charged moieties including, but not limited to, carboxylate, sulfonate, phosphate, or quaternary amine. The charged moiety, in some embodiments, can be located on a group pendant to the block copolymer backbone. In some embodiments, the charged moiety is part of a monomeric species forming a block of the block copolymer. For example, a hydrophilic block comprises at least one poly(2-oxazoline) including an anionic or cationic moiety. Other cationic or anionic monomers can be employed to form blocks of copolymers described herein.

The hydrophobic block may comprise a hydrophobic poly(2-oxazoline) or poly(2-oxazine). Examples of hydrophilic poly(2-oxazoline)s include, without limitation, 2-methyl-2-oxazoline, 2-ethyl-2-oxazoline, and mixtures thereof. The degree of polymerization may vary between 5 and 500. Examples of the hydrophobic polymer block include poly(2-oxazoline)s or poly(2-oxazine)s that are derived from 2-oxazolines or 2-oxazines with hydrophobic substituents at the 2-position of the oxazoline or oxazine ring. In a particular embodiment, the hydrophobic substituent is an alkyl or an aryl. In another embodiment, the hydrophobic substituent comprises 3 to about 50 carbon atoms, 3 to about 20 carbon atoms, 3 to about 12 carbon atoms, particularly 3 to about 6 carbon atoms, or 4 to about 6 carbons. In some preferred embodiments the hydrocarbon groups are aromatic such as unsubstituted or substituted phenyl, benzyl, or the like. In a particular embodiment, hydrophobic polymer blocks are poly(2-propyl-2-oxazoline), poly(2-isopropyl-2-oxazoline), poly(2-butyl-2-oxazoline), poly(2-sec-butyl-2-oxazoline), poly(2-isobutyl-2-oxazine), poly(2-tert-butyl-2-oxazoline), poly(2-pentyl-2-oxazoline), poly(2-hexyl-2-oxazoline), poly(2-heptyl-2-oxazoline), poly(2-octyl-2-oxazoline), poly(2-nonyl-2-oxazoline), poly(2-decyl-2-oxazoline), poly(2-undecyl-2-oxazoline), and poly(2-dodecyl-2-oxazoline). In a particular embodiment, the hydrophobic block copolymer is 2-butyl-2-oxazoline, 2-propyl-2-oxazoline, or mixtures thereof. The hydrophobic block may consist of 1-300 monomer units. In a particular embodiment, the ratio of repeating hydrophilic units to repeating hydrophobic units (in terms of the numbers of repeating units) typically ranges from about 20:1 to 1:2, e.g., from about 10:1 to 1:1, e.g., from about 7:1 to 3:1.

In some embodiments, the hydrophilic poly(2-oxazoline) block is of the formula (I):

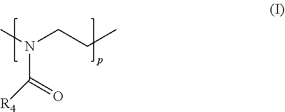

wherein R⁴ is selected from the group consisting of alkyl, cycloalkyl and alkenyl, each optionally substituted with hydroxyl, —SH, —COOR⁵, —NR⁶₂, —CONR⁷ or —CHO, wherein R⁵-R⁷ are independently selected from the group consisting of hydrogen and alkyl and wherein p is at least 5. In some embodiments, for example, the hydrophilic poly(2-oxazoline) block comprises poly(2-methyl-2-oxazoline) or poly(2-ethyl-2-oxazoline). Moreover, the block copolymer may further comprise a second block that is more hydrophobic than the hydrophilic poly(2-oxazoline) block.

In some embodiments, the block copolymer comprises at least one repeating unit (block) of formula (I):

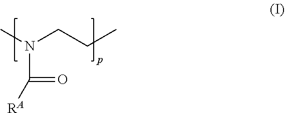

wherein R^{A} is a hydrocarbon group, optionally substituted with —OH, —SH, —COOH, —NR′₂, —COOR′, —CONR′, or —CHO, with R′ representing H or C₁₋₃ alkyl, and with R^{A} being selected such that the repeating unit of formula (I) is hydrophilic.

The block copolymer may also comprise at least one repeating unit (block) of formula (II) or (III):

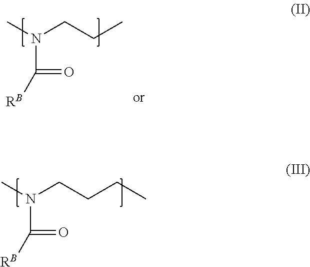

wherein R^{B} is a hydrocarbon group optionally substituted with halogen, —OH, —SH, —COOH, —NR″₂, —COOR″, —CONR″, —CHO, with R″ representing H, alkyl or alkenyl, and with R^{B} being selected such that the repeating unit of formula (II) or (III) is more hydrophobic than the repeating unit of formula (I).

In some embodiments, R^{A} is selected from a C₁₋₈ hydrocarbon group, e.g., a C₁₋₆ hydrocarbon group, e.g., a C₁₋₃, e.g., a C₁₋₂ hydrocarbon group, all of which may be optionally substituted. In certain embodiments the hydrocarbon groups are alkyl groups.

As will be understood, the hydrophilic property of the unit of formula (I) as defined above will depend on the size of the hydrocarbon group in R^{A}. If a small hydrocarbon group is selected, such as methyl or ethyl, the resulting group R^{A}, unsubstituted or substituted with the above substituents, will always be hydrophilic. If a larger hydrocarbon group is selected, the presence of substituents may be advantageous to introduce additional polarity to the unit of formula (I). In some embodiments, R^{A} is selected, independently for each occurrence, from methyl and ethyl optionally substituted with halogen, —OH, —SH, —COOH, —NR′₂, —COOR′, —CONR′, or —CHO, with R′ representing H or C₁₋₃ alkyl, e.g., R^{A} is selected from methyl or ethyl.

In the units of formula (II) or formula (III), R^{B} is a hydrocarbon group optionally substituted with halogen, —OH, —SH, —COOH, —NR″₂, —COOR″, —CONR″, or —CHO, with R″ representing H, alkyl or alkenyl, and with R^{B} selected such that the repeating unit of formula (II) is more hydrophobic than the repeating unit of formula (I). If R″ is alkyl or aryl, in some embodiments R″ is a C₁₋₈ alkyl or aryl group. In some embodiments, halogen substituents, if present, may be selected from Cl and F.

In some embodiments, the block copolymer comprises: at least one hydrophilic A or A′ block and at least one hydrophobic B or B′ block, wherein said hydrophilic A or A′ block is represented by the structure of formula (I) wherein R^{A} is a hydrocarbon group, optionally substituted with —OH, —SH, —COOH, —NR′₂, —COOR′, —CONR′, or —CHO, with R′ representing H or C₁₋₃ alkyl, with R^{A} being selected such that the repeating unit of formula (I) is hydrophilic; and said hydrophobic B block is represented by the structure of formula (II) or (III) wherein R^{B} is a hydrocarbon group selected such that the repeating unit of formula (II) or (III) is more hydrophobic than the repeating unit of formula (I), wherein the hydrocarbon group R^{B} is selected from the group consisting of linear or branched alkyl or aryl (aromatic) hydrocarbons of from least three to thirty carbon atoms (C₃ or C₃₀).

In some embodiments, R^{B} is selected from a C₃₋₂₀ hydrocarbon group, e.g., a C₃₋₁₂ hydrocarbon group, e.g., a C₃₋₆, e.g., a C₄₋₆ hydrocarbon group, all of which may be optionally substituted. In some embodiments, the hydrocarbon group does not carry a substituent.

In some embodiments, the hydrocarbon groups are aliphatic or aromatic groups, such as alkyl groups, aryl groups or alkaryl groups. In some embodiments, the hydrocarbon groups are alkyl groups, such as propyl, isopropyl, butyl, sec-butyl, isobutyl, tert-butyl, pentyl, hexyl, heptyl, octyl nonyl, decyl, undecyl, or dodecyl, or any of their isomers, e.g., C₄₋₆ alkyl groups, i.e., butyl, pentyl, hexyl, e.g., butyl groups, particularly n-butyl and n-nonyl or their structural isomers.

If necessary, i.e., if it is not readily apparent from the chemical structure of R^{A} and R^{B} that a specific unit of formula (II) or formula (III) is more hydrophobic than a given unit of formula (I), this can be verified, e.g., by preparing comparable homopolymers of the respective units and determining their log P value under the same conditions. The log P value, as commonly known, is the logarithm of the partition coefficient observed for a species A between water and n-octanol. In particular, the partition coefficient P of a species A is defined as the ratio P=[A]_{n-octanol}/[A]_{water}, wherein [A] indicates the concentration of A in the respective phase. The more hydrophilic substance will have higher concentrations in water. Typically, the volumes of water and octanol are the same for the measurement.

In some embodiments, the correct selection of R^{B} to provide units of formula (II) or formula (III) which are more hydrophobic than those of formula (I) can be verified by determining the critical micelle concentration (CMC) of a copolymer containing these units according to the procedure disclosed in detail below. If a CMC can be observed, the requirement regarding the hydrophilic nature of the units of formula (I) and the more hydrophobic/less hydrophilic nature of the units of formula (II) is reliably fulfilled.

The requirement that the units of formula (I) are hydrophilic and the units of formula (II) or formula (III) are more hydrophobic than those of formula (I) will also be reliably fulfilled for any possible combination of the following embodiments of R^{A} and R^{B}, as will be apparent from the structures thereof. Namely, R^{A} is selected from methyl and ethyl optionally substituted with —OH, —SH, —COOH, —NR′₂, —COOR′, —CONR′, or —CHO, with R′ representing H or C₁₋₃ alkyl, e.g., R^{A} is selected from methyl or ethyl; and R^{B} is selected from an unsubstituted C₃₋₂₀ hydrocarbon group, e.g., a C₃₋₁₂ hydrocarbon group, e.g., a C₃₋₆, e.g., a C₄₋₆ hydrocarbon group, for example aliphatic or aromatic groups, such as alkyl groups, aryl groups or alkaryl groups. In some embodiments the hydrocarbon groups are alkyl groups, such as propyl, butyl, pentyl, hexyl, heptyl, octyl or nonyl, e.g., C₄₋₆ alkyl groups, i.e., butyl, pentyl, hexyl, e.g., butyl, particularly n-butyl.

A block copolymer comprising repeating units of formula (I) and (II) or (III) above can be conveniently prepared via ring opening polymerization of 2-substituted 2-oxazolines (or 2-substituted 4,5-dihydro oxazoles according to IUPAC nomenclature). Therefore, the polymers used in the context of the invention are also referred to as poly(2-oxazoline)s.

The copolymer according to the invention can comprise other repeating units in addition to repeating units (I) and (II) or (III) above. However, in some embodiments the major portion of all repeating units, i.e., more than 50%, e.g., more than 75%, e.g., more than 90%, e.g., 100%, based on the total number of repeating units, are repeating units of formula (I) or (II) or (III) as defined above. It will be understood that all repeating unit of formula (II) or (III) contained in the copolymer will be more hydrophobic than any of the repeating units of formula (I) contained in the copolymer.

The ratio of repeating units (I) to repeating units (II) or (III), in terms of the numbers of repeating units, typically ranges from 20:1 to 1:2, e.g., from 10:1 to 1:1, e.g., from 7:1 to 3:1.

With respect to the arrangement of the repeating units (I) and (II) or (III) above, the copolymers according to the invention can be random copolymers, copolymers containing segments of polymerized units of the same type (i.e., segments of units of formula (I) and/or segments of units of formula (II)), gradient copolymers or block copolymers. In one embodiment, the copolymers are block copolymers.

In some embodiments, at least one block A of the block copolymer, e.g., all blocks A in the case of multiple occurrences, is (are) represented by formula (I):

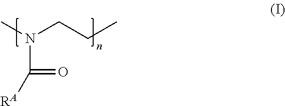

wherein R^{A} represents a methyl or ethyl group, e.g., a methyl group, and n indicates the number of repeating units within the block A. In some embodiments n is an integer of 5 or more, e.g., 10 or more, e.g., 20 or more. It is generally below 300, e.g., 200 or less, e.g., 100 or less, e.g., 50 or less.

In some embodiments, at least one block B of the block copolymer, e.g., all blocks B in the case of multiple occurrences, is (are) represented by formula (III):

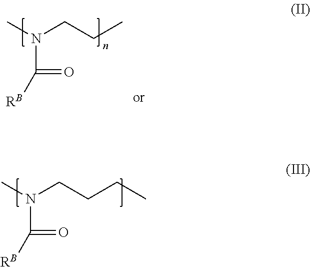

wherein R^{B} is a C₃₋₂₀ hydrocarbon group, e.g., a C₃₋₁₂ hydrocarbon group, e.g., a C₃₋₆ hydrocarbon group, e.g., a C₄₋₆ hydrocarbon group. In some embodiments, the hydrocarbon groups are aliphatic or aromatic groups, such as alkyl groups, aryl groups or alkaryl groups. In some embodiments, the hydrocarbon groups are alkyl groups such as propyl, butyl, pentyl, hexyl, heptyl, octyl or nonyl, e.g., C₄₋₆ alkyl groups, i.e., butyl, pentyl, hexyl, e.g., butyl groups, particularly n-butyl. In some embodiments, the variable n represents an integer of 5 or more, e.g., 10 or more. It is generally below 300, e.g., 200 or less or 100 or less, e.g., 50 or less.

The block copolymer used as a drug delivery system in the context of the invention contains at least one block A and at least one block B as defined above. It may contain one or more additional blocks which are different from A or B. However, in some embodiments the block copolymer contains exclusively blocks falling under the definitions of A and B above. In certain embodiments, all repeating units of the block copolymer are repeating units of formula (I) or (II) or (III) above.

As for the arrangement of blocks A and B in the copolymer used in the context of the invention, in some embodiments the structures of the copolymer can be indicated as (AB)ₘ or (BA)ₘ with m being 1, 2 or 3, as ABA, or as BAB. In some embodiments the block copolymer is an AB or BA diblock copolymer or an ABA triblock copolymer.

Thus, in one embodiment of the invention, the polymeric entities of the block copolymer consist of (an) A block(s) consisting of polymerized 2-methyl-2-oxazoline or 2-ethyl-2-oxazoline (also referred to herein as “poly(2-methyl-2-oxazoline) block” or “poly(2-ethyl-2-oxazoline) block”) and (a) B block(s) consisting of polymerized 2-(C₄₋₆ alkyl)-2-oxazoline. In some embodiments, the copolymer comprises (an) A block(s) consisting of polymerized 2-methyl-2-oxazoline or 2-ethyl-2-oxazoline and (a) B block(s) consisting of polymerized 2-butyl-2-oxazoline (also referred to as “poly(2-butyl-2-oxazoline) block”). In certain embodiments the copolymer is an AB or ABA di- or triblock-copolymer of the above constitution.

It will be understood that compositions comprising combinations, e.g., mixtures or blends of two or more different copolymers are also encompassed by the invention, e.g., combinations of copolymers containing different groups R^{A} and or R^{B}, or combinations of copolymers showing different arrangements of their repeating units, e.g., combinations of a random polymer and a block copolymer.

In a particular embodiment of the instant invention, the copolymer of the instant invention is represented by the formula (IV):

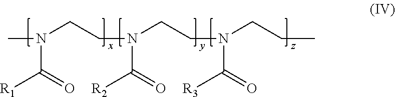

wherein x and y are independently selected between 1 and about 300, particularly about 5 to about 150, and more particularly about 10 to about 100; z is either 0 or from between 1 and about 300, particularly about 5 to about 150, and more particularly about 10 to about 100; R₁ and R₃ are independently selected from the group consisting of —H, —OH, —NH₂, —SH, CH₃, —CH₂CH₃, and an alkyl comprising 1 or 2 carbon atoms; and R₂ is selected from the group consisting of an alkyl or an aryl. In a particular embodiment, x, y, and z are independently 5 or more, 10 or more, or 20 or more, and less than 300, less than 200, less than 100, or less than 50. In a particular embodiment, R₁ and R₃ are independently selected from the group consisting of —CH₃ and —CH₂CH₃. In a particular embodiment, R₂ is the formula (CH₂)ₙR₄, wherein R₄ is —OH, —COOH, —CHCH₂, —SH, —NH₂, —CCH, —CH₃, or —CHO and wherein n is about 2 to about 50, about 2 to about 20, about 2 to about 12, or about 3 to 6. In a particular embodiment, R₂ comprises 3 to about 50 carbon atoms, 3 to about 20 carbon atoms, 3 to about 12 carbon atoms, or 3 to about 6 carbon atoms. In yet another embodiment, R₂ is butyl (including isopropyl, sec-butyl, or tert-butyl) or propyl (including isopropyl). In yet another embodiment, R₂ is —CH₂—CH₂—CH₂—CH₃ or —CH₂—CH₂—CH₃.

The copolymers used in the context of the invention can be prepared by polymerization methods known in the art. For example, poly(2-oxazoline)s can be prepared by living cationic ring opening polymerization. The preparation of random copolymers, gradient copolymers and block copolymers, is described in detail, e.g., by R. Luxenhofer and R. Jordan, Macromolecules 39, 3509-3516 (2006), T. Bonne et al., Colloid. Polym. Sci., 282, 833-843 (2004) or T. Bonne et al. Macromol. Chem. Phys. 2008, 1402-1408, (2007).

Amphiphilic block copolymers can be obtained from hydrophilic 2-methyl-2-oxazoline (MeOx) and hydrophobic 2-nonyl-2-oxazoline (NonOx) (Bonne et al. (2004) Colloid Polym. Sci, 282:833; Bonne et al. (2007) Coll. Polym. Sci., 285:491). Various amphiphilic block copolymers (also additionally bearing carboxylic acid side chains for micellar catalysis (Zarka et al. (2003) Chem-Eur. J., 9:3228; Bortenschlager et al. (2005) J. Organomet. Chem., 690:6233; Rossbach et al. (2006) Angew. Chem. Int. Ed, 45:1309)) and lipopolymers have been reported and their aggregation behavior in aqueous solution was studied (Bonne et al. (2004) Colloid Polym. Sci., 282:833; Bonne et al. (2007) Coll. Polym. Sci., 285:491). CROP allows for an exact tuning of the hydrophilic-lipophilic balance (HLB) and initiation with a bi-functional initiator allows two step synthesis of triblock copolymers in contrast to the three step synthesis necessary when, e.g., methyltriflate is used as an initiator. This approach has the additional benefit that both polymer termini can be easily functionalized with the same moiety.

The initiators used to generate the copolymers of the instant invention can be any initiator used in the art. Additionally, the termini of the copolymers of the instant invention can be any terminus known in the art. The polymers can be prepared from mono-, bi- or multifunctional initiators (such as multifunctional triflates or multifunctional oxazolines) such as, but not restricted to, methyltriflate, 1,2-bis(N-methyloxazolinium triflate) ethane or pentaerithritol tetrakistriflate. Examples of polymer termini include, for example, —OH, —OCH₃,

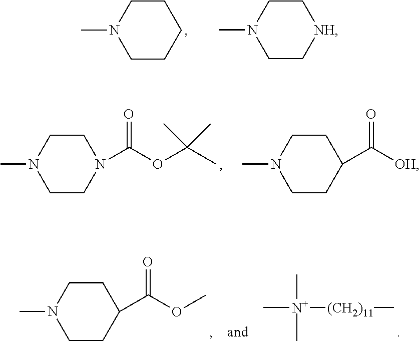

Block copolymers of the instant invention (e.g., piperazine terminated copolymers) may be additionally labeled with a fluorescent dye (e.g., fluorescein isothiocyanate, FITC) to allow evaluation of the localization (e.g., in plasma membrane compartments such as lipid rafts, caveolae, clathrin coated pits) of these polymers by confocal microscopy (Batrakova et al. (2001) J. Pharmacol. Exp. Ther, 299:483; Bonne et al. (2004) Colloid Polym. Sci, 282:833; Bonne et al. (2007) Coll. Polym. Sci, 285:491).

Alternatively, in some embodiments, the second block of the copolymer can include monomer having a side chain comprising a heteroaryl moiety. In some embodiments, the heteroaryl moiety is a five-membered ring or six membered ring. The heteroaryl moiety, in some embodiments, is of the formula:

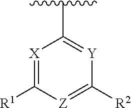

wherein X, Y and Z are heteroatoms independently selected from the group consisting of N, O, and S, and R¹ and R² are independently selected from the group consisting of hydrogen, alkyl, cycloalkyl, alkenyl, aryl, heteroaryl, amine, —SH, hydroxyl, halo, carboxyl, and —CONR³, wherein R³ is alkyl and [symbol] represents a point of attachment of the heteroaryl moiety to the monomer.

In some embodiments, X, Y and Z of the heteraryl moiety are nitrogen to provide a triazine structure. Additionally, R¹ and R² can be selected to further tune the character of the heteroaryl moiety. R¹ and R², for example, can be selected to increase or decrease interactions with compounds carried by micelles formed of the amphiphilic polymer. In some embodiments, R¹ and R² are independently selected from amine functional groups, including primary, secondary and/or tertiary amines. In some embodiments, the second block comprises polymer of the formula (Ia):

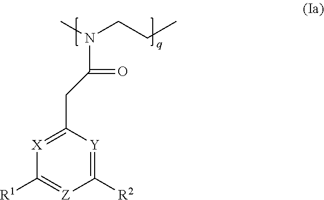

wherein q is at least 5. In some embodiments, q ranges from 10 to 100.

As described herein, the first block of the copolymer can be more hydrophilic or less hydrophobic than the second block. The hydrophilic block can comprise any polymeric species not inconsistent with the technical objectives described herein. In some embodiments, the first block comprises poly(2-oxazoline), including 2-methyl-2-oxazoline, 2-ethyl-2-oxazoline, or combinations thereof. In other embodiments, the first block can comprise polyalkylene oxide, polyesters or polyamide. The first block, in some embodiments, is formed of a single polymeric species, such as poly(2-oxazoline) or other polymer exhibiting greater hydrophilic character relative to the second block. Alternatively, the first block can be formed of two or more polymeric species, such as poly(2-oxazoline) and polyalkylene oxide.

In some embodiments, the first block comprises polymer of the formula (IIa):

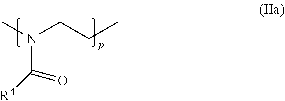

wherein R⁴ is selected from the group consisting of alkyl, cycloalkyl and alkenyl, each optionally substituted with hydroxyl, —SH, —COOR⁵, —NR⁶₂, —CONR⁷ or —CHO, wherein R⁵-R⁷ are independently selected from the group consisting of hydrogen and alkyl, and wherein p is at least 5. In some embodiments, p ranges from 10-300 or 20-200.

In some embodiments, block copolymer described herein is of the formula:

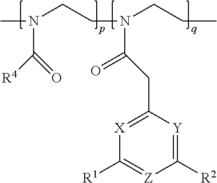

wherein R¹, R², R⁴, X, Y, Z and p and q are described herein. Moreover, amphiphilic block copolymer described herein may also be for the formula:

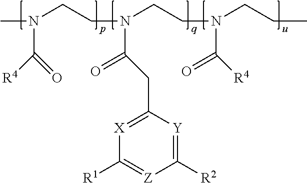

wherein R¹, R², R⁴, X, Y, Z and p and q are described herein, and u ranges from 5-300.

Block copolymers having structure and properties described herein can have any desired ratio of repeating monomer between the first and second blocks. Number of repeating monomeric units in the first block relative to the second block can be selected according to several considerations, including specific chemical structure of the first and/or second blocks, the ability of the block copolymer to form micelles, and/or specific chemical identity of compound(s) to be carried by the micelles. In some embodiments, the ratio of repeating monomer between the first block and second block ranges from 10:1 to 1:2. The ratio of repeating monomer between the first block and the second block, for example, can range from 3:1 to 1:1.

As described herein, the second block can be more hydrophobic than the first block. Hydrophobicity of the second block being greater than the first block may be readily apparent from the specific chemical structures constituting monomer of the first and second blocks. If necessary, i.e., if it is not readily apparent from the chemical structure that specific monomer of the second block is more hydrophobic than monomer of the first block, this can be verified, e.g., by preparing comparable homopolymers of the respective monomers and determining their log P value under the same conditions. The log P value, as commonly known, is the logarithm of the partition coefficient observed for a species A between water and n-octanol. In particular, the partition coefficient P of a species A is defined as the ratio P=[A]_{n-octanol}/[A]_{water}, wherein [A] indicates the concentration of A in the respective phase. The more hydrophilic substance will have higher concentrations in water. Typically, the volumes of water and octanol are the same for the measurement. Additionally, in some embodiments, the differences in hydrophobicity of the first and second blocks can be verified by determining critical micelle concentration (CMC) of the amphiphilic copolymer. If a CMC can be observed, the requirement of the more hydrophobic monomer of the second block and the more hydrophilic monomer of the first block is satisfied.

Block copolymer, in some embodiments, can comprise other repeating units in addition to repeating units of formulas (Ia) and (IIa) above. However, in some embodiments the major portion of all repeating units, i.e., more than 50%, e.g., more than 75%, e.g., more than 90%, e.g., 100%, based on the total number of repeating units, are repeating units of formula (Ia) or (IIa) as defined above. It can be understood, in some embodiments, that all repeating units of formula (Ia) contained in the copolymer will be more hydrophobic than any of the repeating units of formula (II) contained in the copolymer.

As described above, block copolymer described herein can have any desired block structure. Taking the first block as “A” and the second block as “B”, the block copolymer can be indicated as (AB)ₘ or (BA)ₘ with m being and integer from 1 to 10, or as ABA, or as BAB. In some embodiments the block copolymer is an AB or BA diblock copolymer or an ABA triblock copolymer.

In some embodiments, the polymeric entities of the block copolymer consist of (an) A block(s) consisting of polymerized 2-methyl-2-oxazoline or 2-ethyl-2-oxazoline (also referred to herein as “poly(2-methyl-2-oxazoline) block” or “poly(2-ethyl-2-oxazoline) block”) and (a) B block(s) consisting of poly(2-N,N-dimethyl-1,3,5-triazine-2,4-diamine-6-ethyl-2-oxazoline). In certain embodiments the copolymer is an AB or ABA di- or triblock-copolymer of the above constitution. In some embodiments, the second block comprises poly(2-oxazine).

Examples of hydrophilic A blocks (from left to right) are poly(2-methyl-2-oxazoline) and poly(2-ethyl-2-oxazoline):

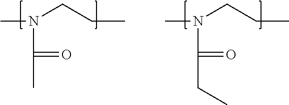

Examples of hydrophobic B blocks (from left to right) are presented below—poly(2-butyl-2-oxazoline), poly(2-butyl-2-oxazine), and poly(2-nonyl-2-oxazoline):

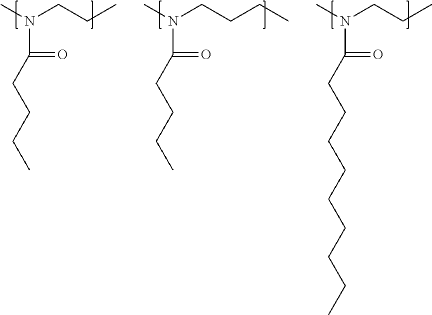

In some examples the block copolymer comprises a hydrophilic A′ block containing carboxylic group such as poly(2-carboxyporyl-2-oxazoline):

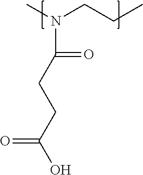

### Lyophilized Powder

In another aspect, lyophilized powders are described herein comprising composite particles, the composite particles including a nanocrystal of an organic compound, and block copolymer associated with one or more surfaces of the nanocrystal of an organic compound, the block copolymer comprising at least one hydrophilic poly(2-oxazoline) block. The composite particles of the lyophilized powder can have any composition and/or properties described hereinabove. Moreover, the composite particles can have an average size of 70-1000 nm, in some embodiments.

In some embodiments, the lyophilized powder may further comprise one or more additives, e.g., known cryoprotectants such as sucrose.

### Pharmaceutical Composition

In some embodiments, a pharmaceutical composition comprises one or more of the composite particles described herein. Preferably, in such compositions, the organic compound of the composite particles may be an active pharmaceutical ingredient (API).

The pharmaceutical compositions according to the invention may optionally be formulated to include one or more composite particles as described herein, together with one or more pharmaceutically acceptable excipients, such as carriers, diluents, fillers, disintegrants, lubricating agents, binders, colorants, pigments, stabilizers, preservatives, and/or antioxidants.

The pharmaceutical compositions comprising the composite particles of the instant invention may be conveniently formulated for administration with any pharmaceutically acceptable carrier(s). For example, they be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the composite particles in the chosen medium may be varied and the medium may be chosen based on the desired route of administration of the pharmaceutical preparation. Except insofar as any conventional media or agent is incompatible with the composite particles to be administered, its use in the pharmaceutical preparation is contemplated.

The pharmaceutical compositions can be formulated by techniques known to the person skilled in the art, such as the techniques published in Remington's Pharmaceutical Sciences, 20th Edition. The pharmaceutical compositions can be formulated as dosage forms for oral, parenteral, such as intramuscular, intravenous, subcutaneous, intradermal, intraarterial, rectal, topical, pulmonary or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for pulmonary administration/pulmonary delivery can be administered via inhalation and insufflation, for example by a metered dose inhaler. Dosage forms for topical administration include creams, gels, ointments, salves, patches and transdermal delivery systems.

In some embodiments including but not limited to aerosols, orally administered liquids and injectable aqueous dispersions it is important to minimize foaming. Thus, the block co polymers of the present invention that have anti-foaming properties are preferred. For example, block copolymers with a foam height (cm) less than 3 cm is preferred and a foam half-life (hr.) less than 3 hr. is preferable.

In some embodiments anti-foaming agents can be added to decrease or eliminate or prevent the foaming as observed by visual inspection, and measuring height of foam after vial shaking for 10 to 30 sec. A defoamer or an anti-foaming agent is a chemical additive that reduces and hinders the formation of foam in industrial process liquids. The terms anti-foam agent and defoamer are used interchangeably. Many antifoaming agents known in the art that are commonly used can be added to prevent or counter the foam generation in the nano-sized composite particle formulation of this invention (see Mousumi KarYashu, Chourasiya Rahul, Maheshwari Rakesh, K. Tekade, Chapter 2—Current Developments in Excipient Science: Implication of Quantitative Selection of Each Excipient in Product Development, Advances in Pharmaceutical Product Development and Research, 2019, Pages 29-83.

### Pharmaceutical Composition is a Dispersion

In some embodiments, the pharmaceutical composition may be a dispersion. In some embodiments, a dispersion comprises an aqueous or aqueous-based continuous phase, and a dispersed phase comprising composite particles, the composite particles including a nanocrystal of an organic compound, and block copolymer associated with one or more surfaces of the nanocrystal of an organic compound, the block copolymer comprising at least one hydrophilic poly(2-oxazoline) block.

The composite particles of the dispersion can have any composition and/or properties described hereinabove. For example, they preferably have a size from 70 nm to 1,000 nm. Preferably, in such compositions, the organic compound of the composite particles may be, but is not limited to, an active pharmaceutical ingredient (API).

In some embodiments, the composite particles of the dispersion maintain a polydispersity index (PDI) of less than 0.3 for at least 5 days. In preferred embodiments, the PDI may remain below 0.3 or below 0.2 for at least 20 days.

In some embodiments, the organic compound (e.g., the API) are present at a concentration of 1-50 mg/mL, 1-40 mg/mL, 1-30 mg/mL, 1-20 mg/mL, 1-10 mg/mL, 1-5 mg/mL. As described further herein, the dispersion can be employed to generate an aerosol.

As discussed above, it is important to minimize foaming in dispersions. Thus, the block copolymers of the present invention that have anti-foaming properties are preferred. For example, block copolymers with a foam height (cm) less than 3 cm and/or a foam half-life (hr.) less than 3 hr. are preferred. Examples of successful anti-foaming agents include oleic acid or poloxamer 181.

Decreasing foaming is critical for ensuing constant mass output rate of the organic compound (e.g., the API) during aerosol production. It was unexpectedly discovered that poly(2-oxazolines) block copolymer of this invention used as stabilizer greatly decrease foaming compared to standard polyethylene glycol (PEG)-containing polymeric surfactants F127. By adjusting the block lengths in poly(2-oxazoline) and poly(2-oxazoline)-poly(2-oxazine) block copolymers, stable nanocrystal suspensions with desired particle size and no foaming or minimal foaming can be formed.

Aqueous or aqueous-based dispersions of composite particles described herein, in some embodiments, can be stable for at least one-hour. The aqueous or aqueous-based dispersions, for example, can be stable for 1-12 hours or 1-24 hours. In some embodiments, the aqueous or aqueous-based dispersions are stable for 1-7 days or more than 7 days. Moreover, the aqueous or aqueous-based dispersions can remain stable through purification and/or storage processes. The stability of the aqueous or aqueous-based dispersions can facilitate stable freezing, lyophilization, and centrifugation, for example.

### Methods of Treating a Patient

In a further aspect, methods of treating patients are provided.

It will be understood that the composite particles described herein are suitable for use in the treatment or prevention of a wide variety of diseases or disorders such as cancer, neurodegenerative diseases, hepato-biliary diseases, cardiovascular diseases, or pulmonary diseases. The invention also encompasses the use of the composite particles as described above in the preparation of a pharmaceutical composition for the treatment or prevention of any of these diseases. Moreover, diagnostic applications of the composite particles and compositions comprising them are also envisaged.

The composite particles described herein will generally be administered to a patient as a pharmaceutical composition. These composite particles may be employed therapeutically, under the guidance of a physician. While the therapeutic agents are exemplified herein, any bioactive agent may be administered to a patient, e.g., a diagnostic agent.

The dose and dosage regimen of composite particles according to the invention that are suitable for administration to a particular patient may be determined by a physician considering the patient's age, sex, weight, general medical condition, and the specific condition for which the composite particle is being administered and the severity thereof. The physician may also take into account the route of administration, the pharmaceutical carrier, and the biological activity of the organic compound of the composite particle.

Selection of a suitable pharmaceutical preparation will also depend upon the mode of administration chosen. For example, the composite particle of the invention may be administered by direct injection to a desired site. In this instance, a pharmaceutical preparation comprises the composite particle dispersed in a medium that is compatible with the site of injection.

Composite particles of the instant invention may be administered by any method. For example, they can be administered, without limitation parenterally, subcutaneously, orally, topically, pulmonarily, rectally, vaginally, intravenously, intraperitoneally, intrathecally, intracerebrally, epidurally, intramuscularly, intradermally, or intracarotidly. In a particular embodiment, the composite particles are administered intravenously or intraperitoneally. Pharmaceutical preparations for injection are known in the art. If injection is selected as a method for administering the composite particle, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches, and transdermal delivery systems.

For example, the pharmaceutical compositions according to the invention may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth and/or nose), gastrointestinal, intrauterine, intraocular, subcutaneous, ophthalmic (including intravitreal or intracameral), rectal, and vaginal. Oral and parenteral, especially intravenous administration is generally preferred since the compositions according to the invention provide a sufficient solubility and bioavailability for these routes even when hydrophobic active agents are used.

If the pharmaceutical compositions are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds pharmaceutical compositions, and/or by using infusion techniques. For parenteral administration, the compounds are best used in the form of a sterile aqueous solution which may contain other substances, for example, enough salts and/or glucose to make the solution isotonic with blood. The aqueous solutions should be suitably buffered (e.g., a pH of from 3 to 9), if necessary. The preparation of suitable parenteral formulations under sterile conditions is readily accomplished by standard pharmaceutical techniques well known to those skilled in the art.

The pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (e.g., corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Exemplary excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, the pharmaceutical compositions can be administered in the form of a suppository or pessary, or it may be applied topically in the form of a gel, hydrogel, lotion, solution, cream, ointment or dusting powder. The compositions of the present invention may also be dermally or transdermally administered, for example, by the use of a skin patch.

The pharmaceutical compositions may also be administered by the pulmonary route, rectal routes, or the ocular route. For ophthalmic use, they can be formulated as micronized suspensions in isotonic, pH adjusted, sterile saline, or as solutions in isotonic, pH adjusted, sterile saline, optionally in combination with a preservative such as a benzylalkonium chloride. Alternatively, they may be formulated in an ointment such as petrolatum.

For topical application to the skin, pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax, and water. Alternatively, they can be formulated as a suitable lotion or cream, suspended, or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

Treatment with a Dispersion

In some embodiments, a method of treating a patient involves the use of a dispersion as described herein above. The dispersion, for example, comprises the following: (1) an aqueous or aqueous-based continuous phase, and (2) a dispersed phase comprising composite particles as described herein above. Preferably, in such dispersions, the organic compound of the composite particles may be a pharmaceutical compound, e.g., one exhibiting antiviral properties, including but not limited to, Remdesivir. A therapeutic compound may also be used. In such dispersion, the pharmaceutical compound or therapeutic compound may be present at a concentration of 0.5-50 mg/mL, 1-40 mg/mL, 1-30 mg/mL, 1-20 mg/mL, 0.5-20 mg/mL, 1-10 mg/mL, 1-5 mg/mL.

In some embodiments, the block copolymer is present at a concentration less than 1% by mass of the pharmaceutical or therapeutic compound concentration. The block copolymer, for example, may be present at a concentration of 0.5-5 percent by mass of the therapeutic compound or pharmaceutical compound concentration.

A first step of the treatment may comprise generating an aerosol from the dispersion, e.g., by nebulizing the dispersion. Then, the generated aerosol may be administered to the patient. For example, it may be administered in such a way that the patient can inhale the aerosol. In some embodiments, the patient may be one who is suffering from a respiratory virus, e.g., COVID-19. Suffering from may mean diagnosed with a respiratory virus or presenting symptoms of a respiratory virus, e.g., fever, cough, or difficulty breathing, etc.

In particularly preferred embodiments, when the dispersion is nebulized, about 50% is nebulized into respirable particles, e.g., particles having a size less than 5 microns. These and other embodiments are further illustrated in the following non-limiting examples.

### EXAMPLES

### Examples 1 and 2, and Comparative Example 1

Initial work involved formation and evaluation of the following (1) an inventive composite particle (including Remdesivir as the organic compound/pharmaceutical compound, and P2 as the block copolymer), i.e., Inventive Example 1; and (2) a comparative composite particle (including Remdesivir as the organic compound/pharmaceutical compound, and F127 as the block copolymer), i.e., Comparative Example 1.

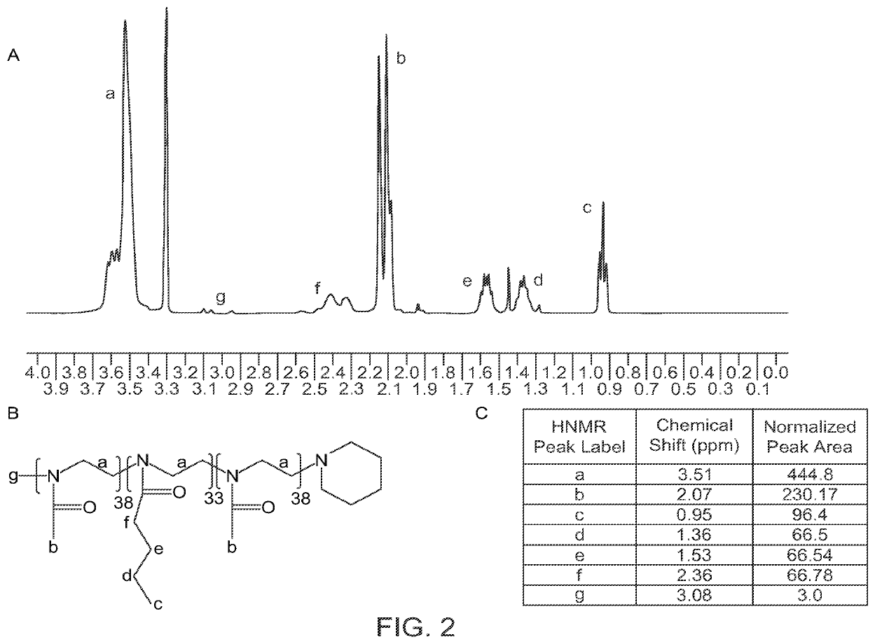

P2 is an A-B-A triblock copolymer comprising a Poly(2-oxazoline) (POx) backbone. The Table in FIG. 1 shows the structure of P2. FIG. 2 includes the proton nuclear magnetic resonance imaging (¹H-NMR) confirmation of structure. It has two flanking hydrophilic 2-methyl-2-oxazoline (MeOx) A blocks and a central, hydrophobic 2-n-butyl-2-oxazoline (BuOx) B block: P[MeOx-b-BuOx-b-MeOx]. Notably, the MeOx block is more hydrophilic than poly(ethylene oxide) (PEO) in F127, and the BuOx block contains both hydrophobic as well as polar moieties that are capable of ion-dipole interactions and forming hydrogen bonds with drug molecules. It is posited that the P2 could adsorb at the surface of growing Remdesivir nanocrystals with the aid of the BuOx block, while the MeOx block could form a protective hydrophilic brush stabilizing the nanocrystals in dispersion.

F127 is an amphiphilic A-B-A triblock copolymer consisting of flanking hydrophilic blocks of PEO and a central hydrophobic poly(propylene oxide) (PPO) block. PEO is also known as poly(ethylene glycol) (PEG). A structure and HLB value for F127 is found in FIG. 1.

Preparation of P2 (Inventive Example 1) and F127 (Comparative Example 1) Composite Particles with Remdesivir as the Organic Compound/Pharmaceutical Compound

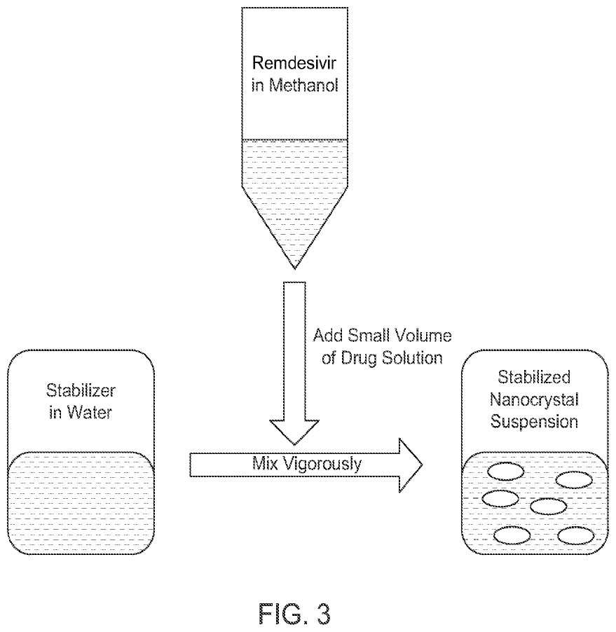

To prepare P2 or F127 stabilized Remdesivir nanocrystals, P2 or F127 were dissolved in water at concentrations ranging from 0.0 to 0.25 mg*mL⁻¹ (which corresponds to 0-5% by weight of Remdesivir e.g. 5% P2 or F127 is 0.25 mg*mL⁻¹ P2 or F127 compared to 5 mg*mL⁻¹ Remdesivir). Remdesivir in methanol (80 mg*mL⁻¹) was then added to the solution to make it 5.0 mg*mL⁻¹ in 2 mL water. This synthetic procedure is depicted graphically in FIG. 3, where the stabilizer is block copolymers P2 or F127. Solutions were stirred rapidly, and the growth of the nanocrystals was monitored over time by Dynamic Light Scattering (DLS).

### Results for F127-Stabilized Remdesivir (Comparative Example 1)

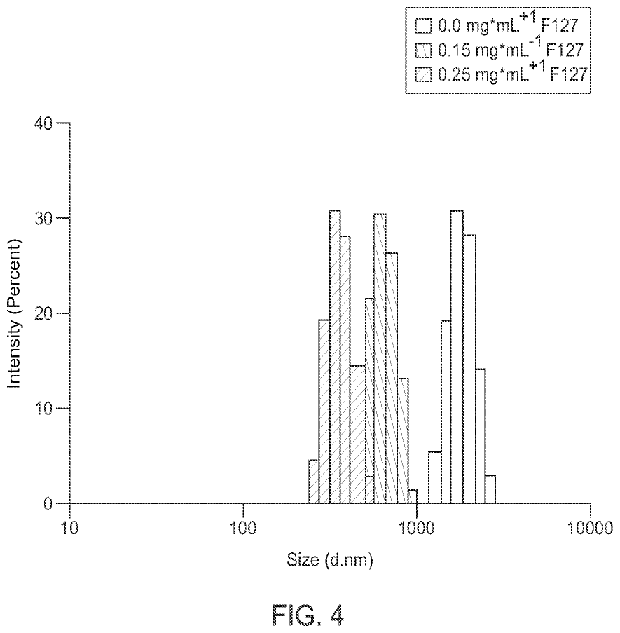

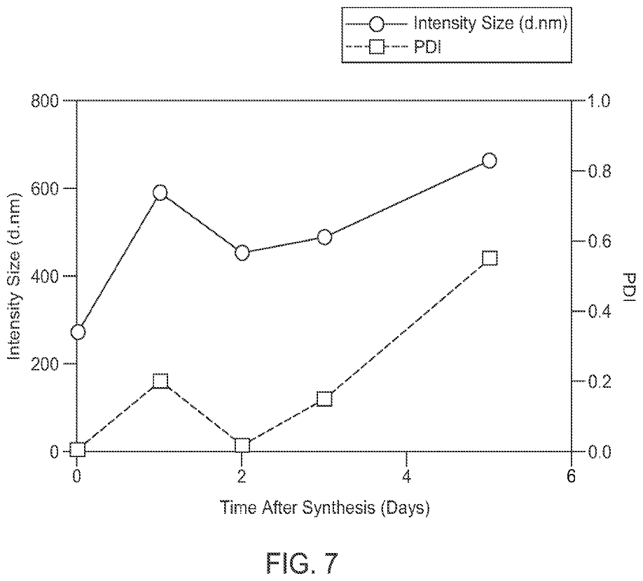

FIG. 4 shows the effect of the starting stabilizer concentration on the size of the Remdesivir nanocrystals. As expected, the smaller quantities of F127 yielded higher nanocrystal sizes as there were more Remdesivir molecules per molecule of F127. In the absence of F127, large multi-micron sized particles formed immediately and irreversibly precipitated out of solution soon after. In the presence of 0.25 mg*mL⁻¹ F127 (5% of Remdesivir by weight), the nanocrystals formed with effective diameter of 341.1 nm, as measured by DLS. See FIG. 4. The stability of these nanocrystals was monitored over a period of 5 days where their size distribution shifted towards larger particles. This may be explained by an Ostwald ripening process. Eventually, they aggregated completely and precipitated out of solution irreversibly after the 5^{th} day (could not be measured on DLS). See FIG. 7.

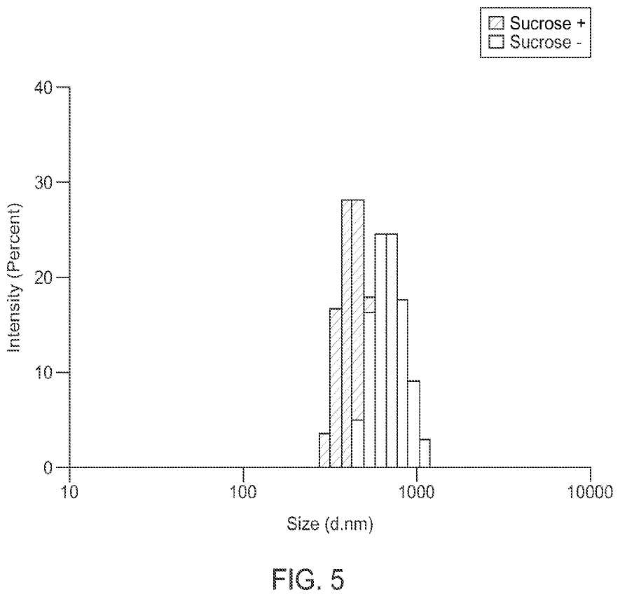

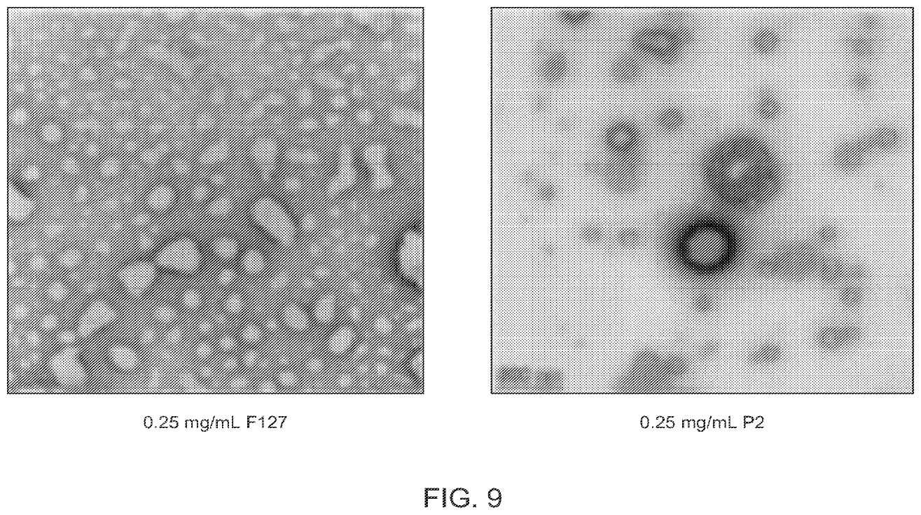

The methanol used precludes use as a therapeutic. To eliminate methanol from the solution, and perhaps improve stability, the composition was lyophilized with and without 9% sucrose and then rehydrated the powders with ultrapure water to the same original volume of the lyophilized aliquot. Although the particles in the sample lyophilized with sucrose were smaller than those formed in the absence of sucrose (FIG. 5) they were stable in dispersion for only 30 min after hydration. TEM of the F127 stabilized nanocrystals is seen in FIG. 9.

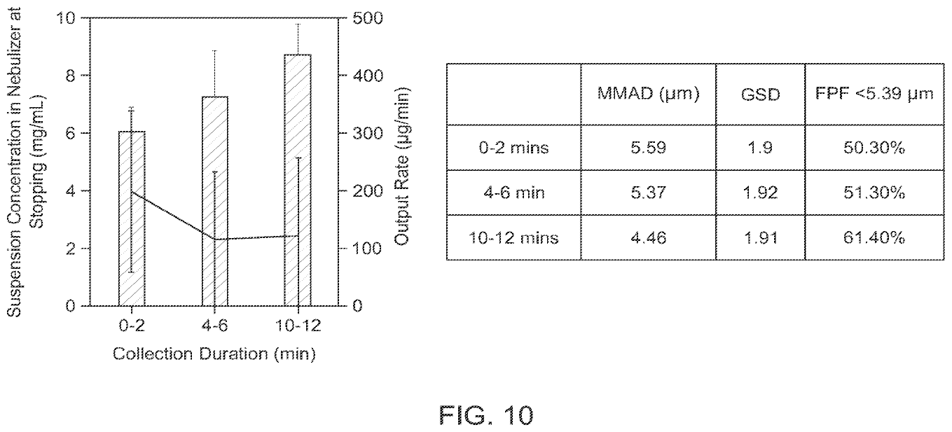

Some samples of these F127 nanocrystals were prepared and tested as an aerosol immediately post hydration (FIG. 10). Solution foaming led to inconsistent drug output rates over 12 min. (See left-hand graph in FIG. 10). Despite these inconsistent output rates, the mass median aerodynamic diameter (MMAD) remained below or near 5 μm (See right-hand table in FIG. 10), which is at the upper end of what is generally considered respirable (typically defined as particles with MMAD 1-5 μm). To increase the consistency of drug output, additional combinations of stabilizers by adding Tyloxapol, oleic acid, and Pluronic L61 in select combinations which could provide anti-foaming effects. These substances offered no additional nanocrystal stability to enhance the long-term storage needed for clinical translation. Solutions still precipitated soon after being hydrated post-lyophilization. Ultimately, the stability of the F127 stabilized nanocrystals (Comparative Example 1) were found to be unsatisfactory due to their lack of long term stability in dispersion.

### Results for P2 Stabilized Remdesivir (Inventive Example 1)

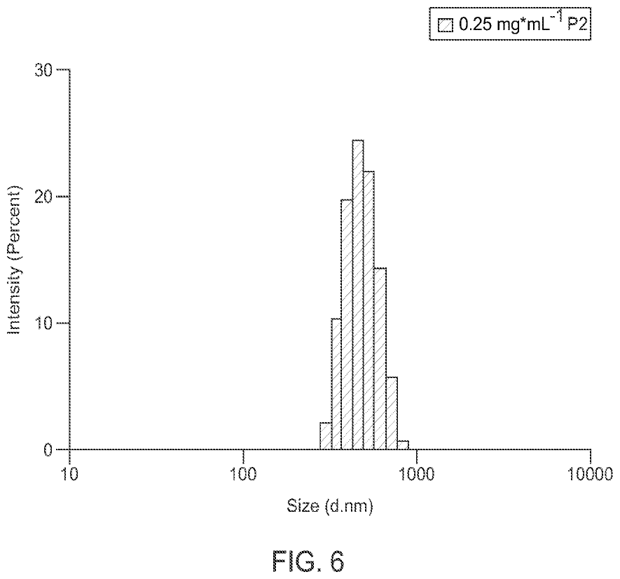

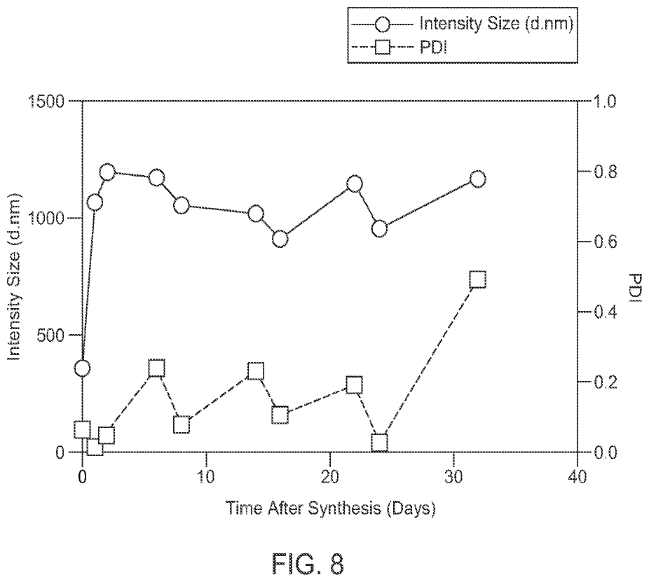

The nanocrystals formed in the presence of 0.25 mg*mL⁻¹ P2 were larger than those synthesized with F127 (see FIG. 6), but were stable in solution for greater than 25 days after synthesis (see FIG. 8). TEM of P2 stabilized nanocrystals is seen in FIG. 9.

### Optimization of P2 Stabilized Remdesivir (Inventive Example 2)

After determining that the P2 stabilized nanocrystals could be prepared at suitable sizes for aerosol delivery, with minimal excipient, and nebulized with minimal foaming, evaluation of the stability of the solution during the process of methanol removal began. One small change to the synthetic procedure depicted in. In the new procedure, both Remdesivir and P2 were mixed in methanol and add the solution to ultrapure water under mixing. Specifically, the method is as described herein below in the Experimental Section. FIG. 3.

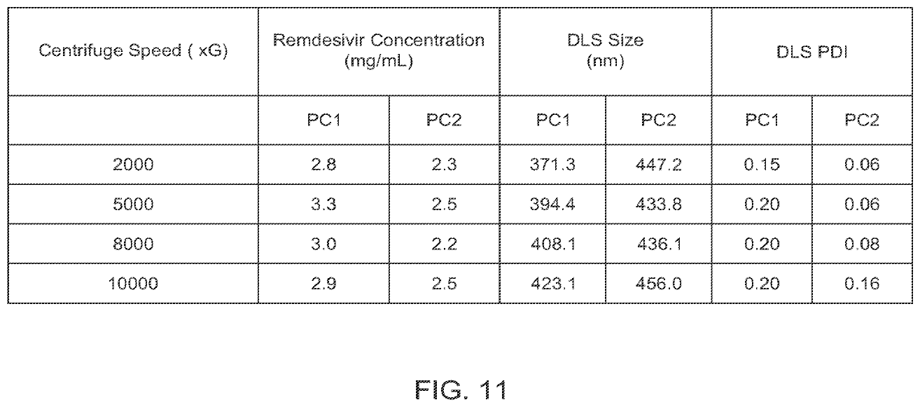

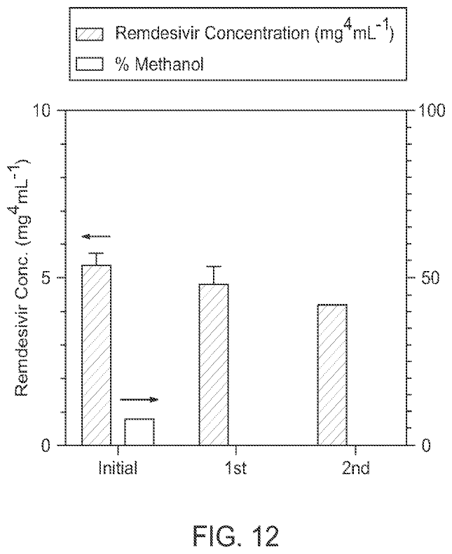

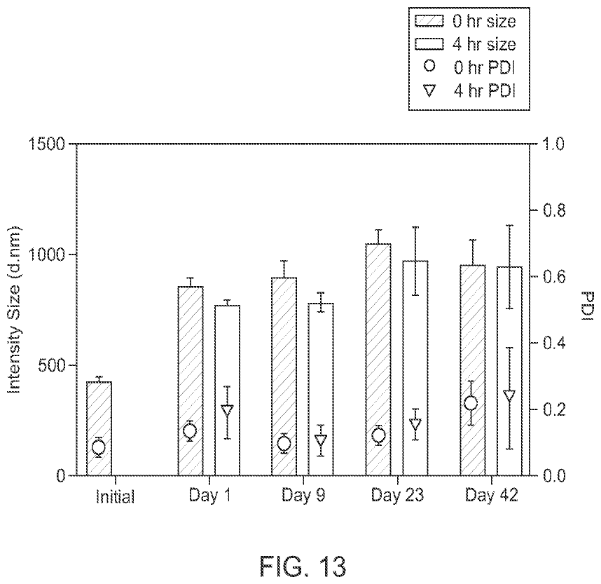

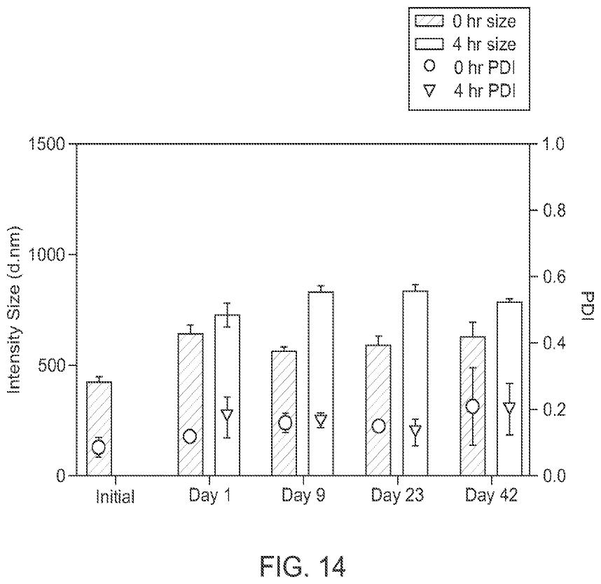

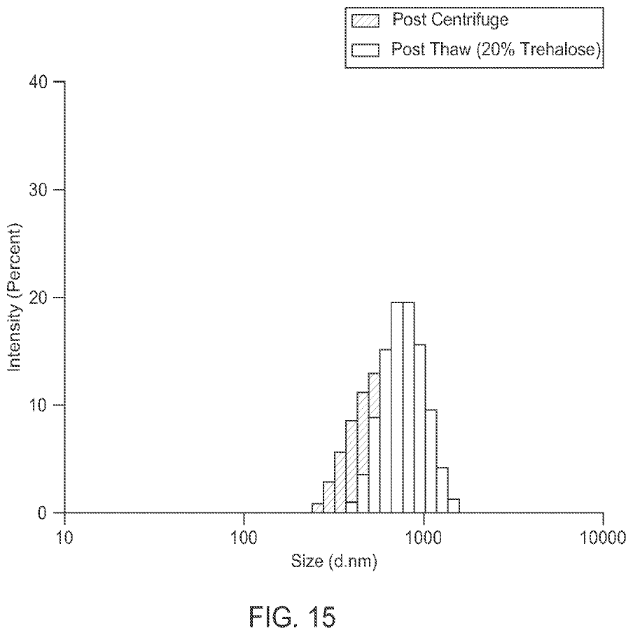

At this point, the feasibility of multiple storage conditions for the P2 stabilized nanocrystals were evaluated, namely, freezing and lyophilization. For the freezing process, methanol was removed by a series of centrifugations. Remaining methanol was quantified using ¹HNMR spectroscopy. Additionally, size and PDI of the nanocrystals after a first and second centrifugation were monitored and noted in the Table in FIG. 11 that the size was maintained mostly in the 400-450 nm range prior to freezing and a low PDI of about 0.20 and below was maintained as well. Nanocrystals were readily resuspended into solution after pelleting indicating their stability through this process. From this, it was concluded that the nanocrystals were stable through centrifugation steps. FIG. 12 shows that methanol can be removed from the formulation by this process while retaining Remdesivir. After just a single centrifuge, the resuspended nanocrystals were less than 0.002% methanol by volume and 4.9 mg*mL⁻¹ Remdesivir (initially 5.4 mg*mL⁻¹). The size is about 450 nm after the centrifugation and resuspension (FIG. 13). Once methanol was removed, the formulations were frozen in liquid nitrogen with 20% trehalose (w/v) as a cryoprotectant where they could be stored long term. After thawing there was a modest increase in the nanocrystal size when thawed between 1 and 42 days after synthesis (FIG. 14). Suspension size was measured at 0 hrs. and 4 hrs. after thawing and there was a trend of increasing size over this time, but no changes in PDI were observed. In comparison to lyophilization (FIG. 13), the initial sizes were smaller when frozen rather than lyophilized. However, no further growth occurred during the 4 hours post-hydration when lyophilized. It was concluded that both freezing and lyophilization were valid methods for long term storage of these suspensions, though they achieved slightly different terminal nanocrystal sizes. It was opted to proceed using the freeze/thaw technique for long-term storage. Size increases were modest over 4 hours, and the crystals were of smaller size when frozen and thawed 550-600 nm compared to 750-950 nm when lyophilized. To thaw, nanocrystals were left at room temperature then shaken gently every few minutes for 15 min. At this point, the nanocrystal size was stable and nanocrystal suspension was ready for size measurements and to use for aerosol and other studies (FIG. 15). To bypass the centrifugation and lyophilization steps, the use of ethanol as an organic solvent to inject Remdesivir into the aqueous media was assessed. Ethanol is employed in commercially available pharmaceutical aerosol formulations so complete removal is not as critical. Unfortunately, ethanol caused the nanocrystals to precipitate quickly and irreversibly, perhaps due to increased hydrophobic interactions, so ethanol was not explored further as a solvent for the nanocrystal preparation.

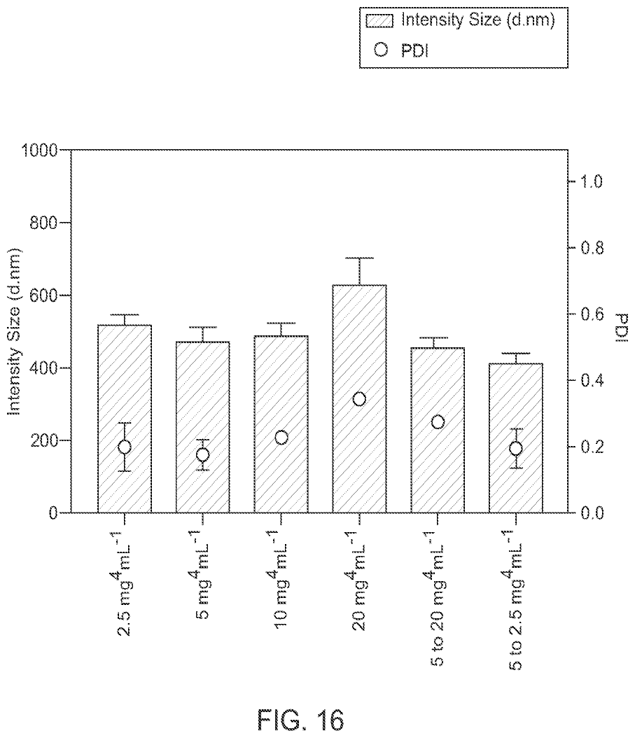

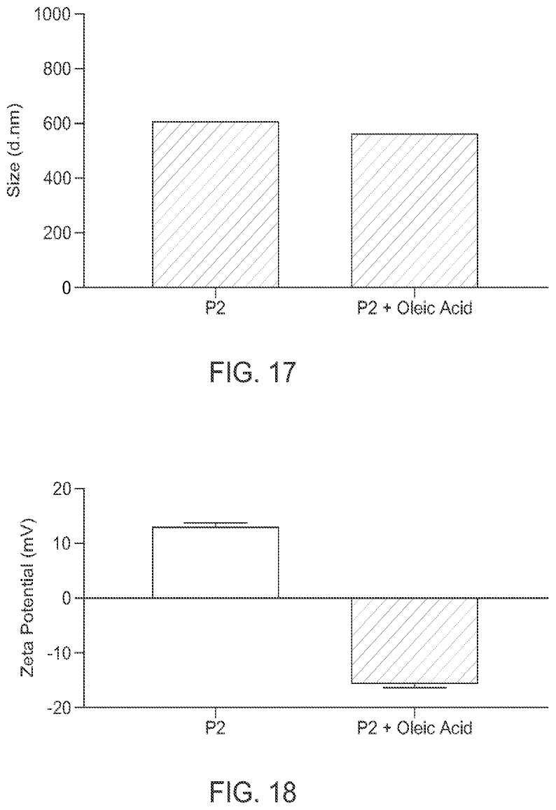

Additionally, FIG. 16 shows how the concentration of Remdesivir at synthesis does not affect the terminal size of the nanocrystals after freeze/thaw. Crystals were prepared at concentrations ranging from 2.5 mg*mL⁻¹ to 20 mg*mL⁻¹ and the sizes remained in a narrow range from 475 to 632 nm. The PDI was slightly increased at the 20 mg*mL⁻¹ concentration, so the ability to concentrate and dilute after a 5 mg*mL⁻¹ synthesis was tested. Sizes and PDI were reduced when concentrating or diluting after centrifugation and prior to freezing. For certain applications, the addition of charged stabilizers to confer zeta potential to the particles could be advantageous. To this end, a small amount (0.005 mg*mL⁻¹, 5 mg*mL⁻¹ Remdesivir) of oleic acid as an additional stabilizer was added. While addition of oleic acid it did not change the terminal size after freeze/thaw (FIG. 17), it did confer a significant negative zeta potential to the nanocrystals (FIG. 18). P2 stabilized nanocrystals had a positive zeta potential, while the addition of oleic acid rendered them negatively charged. Altering the particle surface charge and zeta potential in the future could be useful for improved stability, changing nanoparticle binding with/penetration of mucus, and ultimately internalization into cells like type II pneumocytes which play a critical role in COVID-19 pathogenesis. This introduces a degree of tunability as needed for use in vivo.

### Dissolution Behavior and In Vitro Method Evaluation of P2 Remdesivir Nanocrystals (Inventive Example 2)

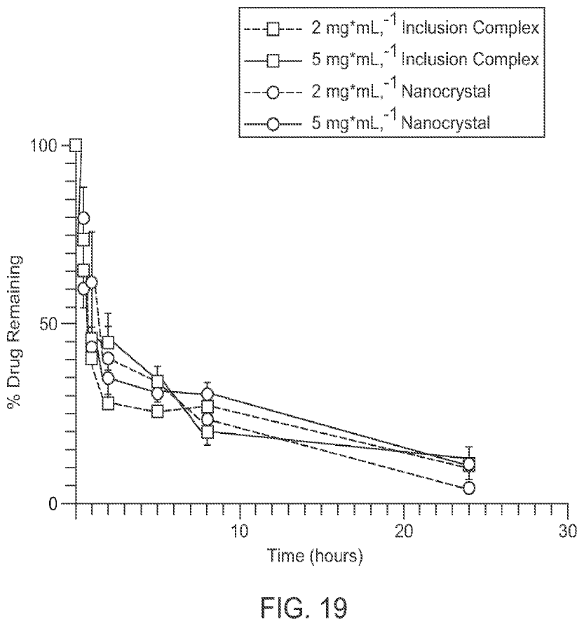

After establishing a final formulation of P2 stabilized nanocrystals at the 5% stabilizer relative to 95% Remdesivir ratio (at any Remdesivir concentration) using stir bar mixing at room temperature, in vitro characteristics of the formulation were evaluated. Most nebulizer reservoirs can accommodate 6 mL of formulation. At 10-20 mg doses, approximately 1.5 to 5 mg*mL⁻¹ concentrations would be needed to deliver adequate drug (this is 5-10% of IV dose). For in vitro drug release study, it was elected to use PBS at 37° C. under sink conditions. Albumin, a major drug binding component of plasma, is not a major component of pleural fluid in the lungs making it unnecessary to include it in the release medium. To compare, a SBECD based cyclodextrin inclusion complex (IC) to mimic Gilead's commercial Remdesivir formulation was prepared. Gilead's formulation contains a 1:30 ratio of Remdesivir to SBECD stabilizer at 5 mg*mL⁻¹ Remdesivir concentration (150 mg*mL⁻¹ SBECD, 96.8% excipient). Both formulations, nanocrystal and IC, were analyzed for drug release at 2 and 5 mg*mL⁻¹ initial concentrations (FIG. 19). All formulations had nearly identical drug release kinetic profiles indicating that the nanocrystals, like the ICs, are simple solubilizing formulations. However, the nanocrystals can solubilize Remdesivir at much lower excipient concentration than the SBECD based formulation (5% formulation weight POx compared to 96.7% formulation weight SBECD).

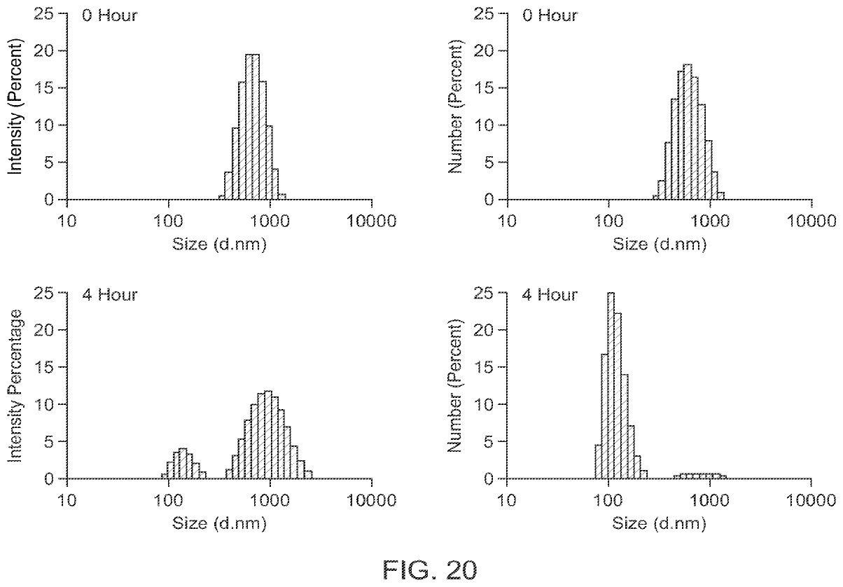

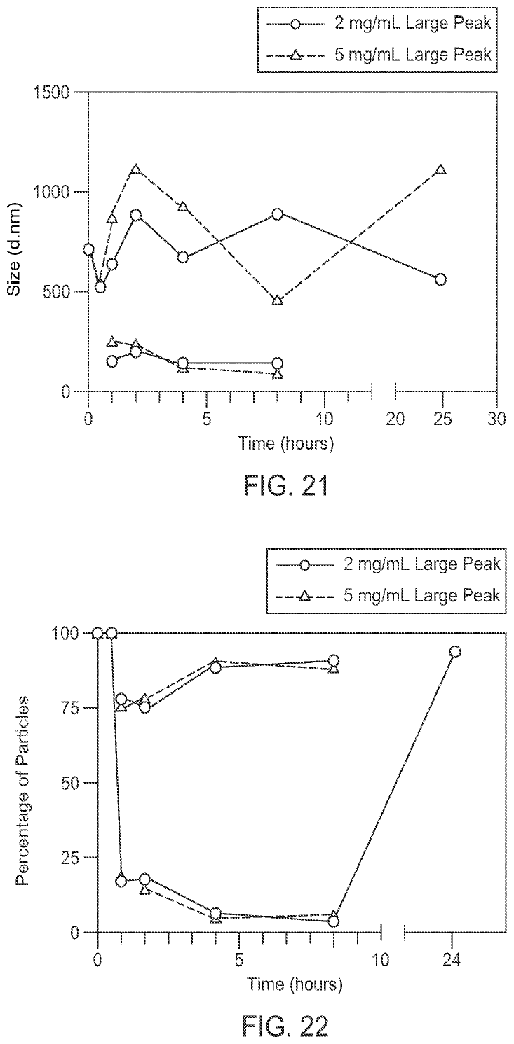

Changes in the particle size in the process of the drug release from the P2 stabilized nanocrystals was measured. The initial nanocrystals at both drug concentrations had approximately the same size, about 750 nm (FIG. 20 (top)). Subsequently, the nanocrystals began to separate into smaller nanoparticles of roughly 150-200 nm as seen in FIG. 20, with the initial nanocrystals disappearing. Notably, on the number size distribution in FIG. 20, almost the entire particle population consists of the smaller 150-200 nm particles at 4 hours. This smaller population first appeared after around 1 hour of drug release and encompassed about 80% of the crystals present (FIG. 21 and FIG. 22). Up until 8 hours, this population grew until it encompassed 95% of all crystals in solution. After 24 hours, when just about 10% of the drug remained in both the nanocrystal and SBECD formulations, the population had reverted to just a single peak. It is hypothesized that upon dilution, the 750 nm nanocrystals break up into smaller aggregates which have a larger surface area/volume ratio. At later time points, only the larger crystals remain as most of the smaller ones have dissolved.

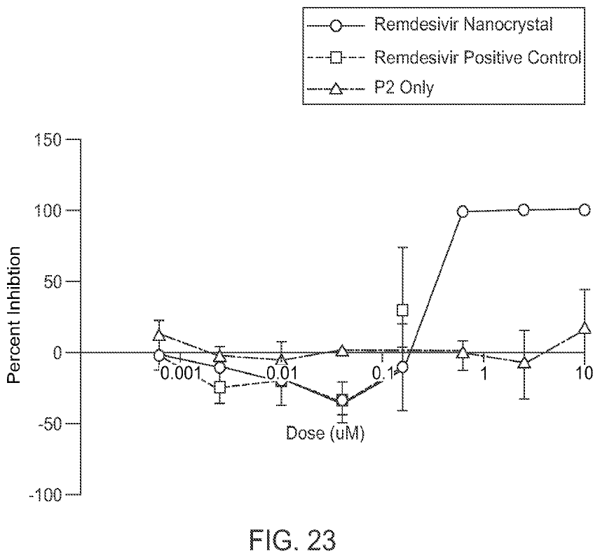

After demonstrating that the nanocrystals have a similar release profile to a commercial Remdesivir IC formulation mimic, the nanocrystal formulation anti-viral efficacy (IC50 Comparison) was assessed. Virus replication was monitored using SARS-CoV-2 virus that expresses nanoluciferase in place of ORF7a. Nanoluciferase expression as measured by luminescence correlates with virus replication. Replacement of ORF7a with a reporter protein does not impact virus replication and allows for high throughput screening of compounds for antiviral activity. A549 cells (human alveolar epithelial cells) expressing Ace2, a prominent receptor for SARS-CoV-2, are pre-treated with formulations at varied concentrations followed by infection with the SARS-CoV-2 reporter virus. After 48 hours, nanoluciferase expression is assayed and normalized to expression observed in DMSO treated controls. FIG. 23 demonstrates that the nanocrystals maintain similar efficacy to the control free Remdesivir making them suitable candidates for aerosol delivery directly to the lungs.

### Aerosol Characterization of P2 Remdesivir Nanocrystals

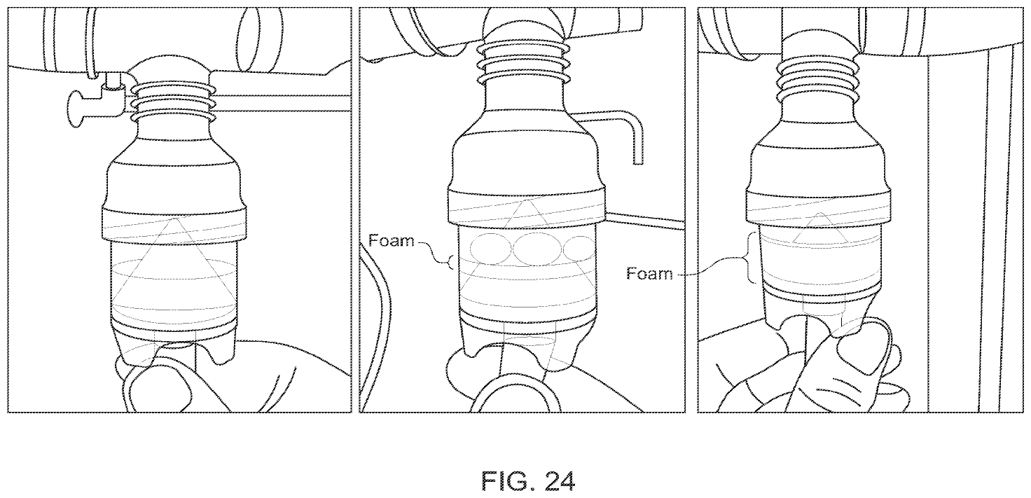

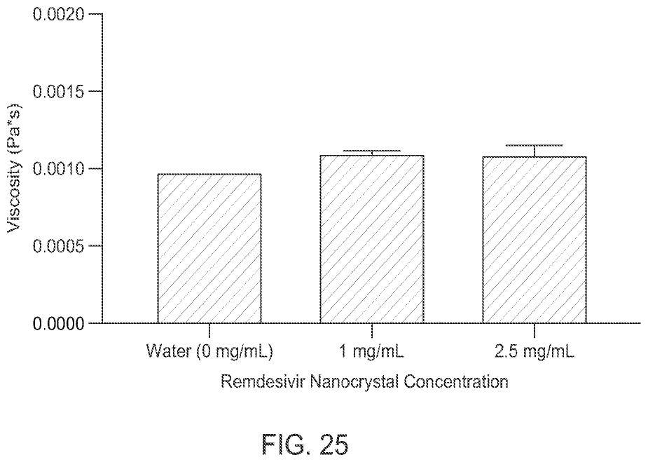

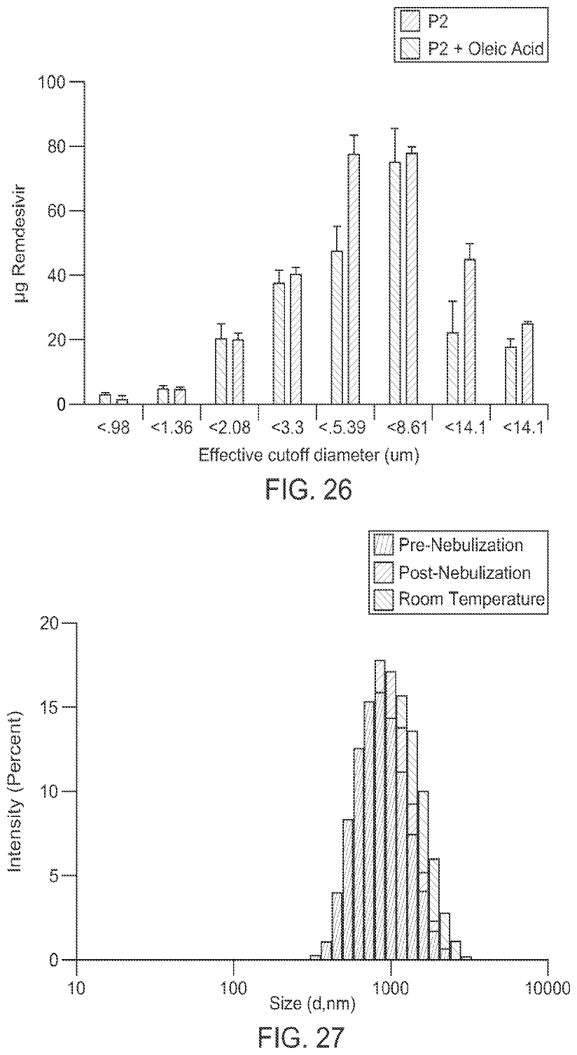

Finally, the ability of the formulation to be aerosolized into respirable particles using an air jet nebulizer was analyzed. An air jet nebulizer was chosen rather than a vibrating mesh nebulizer to facilitate the delivery of a drug suspension. The Nebutech 8960 was selected as a simple, inexpensive device which is readily available to respiratory therapists. It reflects the urgent need to adopt a simple and inexpensive solution for a potentially large number of patients. Using the 10 mg*mL⁻¹ frozen formulation, it was thawed and diluted to a concentration of 1.5 mg*mL⁻¹ Remdesivir. The aerosol particle output of the nebulizer was analyzed from 0 to 2 min. FIG. 24 shows how the choice of excipient can have significant impact on the foaming of the solution in the nebulizer. Generally, foaming leads to inconsistent output over time and should be avoided. The P2 stabilized nanocrystals had minimal foaming when compared to just water. On the other hand, significant foaming was evident (FIG. 24 (right-hand image)) in the F127 stabilized nanocrystals (Comparative Example 1) which led to the inconsistent output over time as shown in FIG. 10. Another factor effecting aerosol particle output is the solution viscosity. FIG. 25 shows there was no major difference in viscosity comparing water to the 2.5 mg*mL⁻¹ Remdesivir nanocrystal suspension (0.125 mg*mL⁻¹ P2). After being placed in the nebulizer and aerosolized for two minutes, the P2 stabilized nanocrystals exhibited little if any change in the particle size compared to the initial suspension or the nanocrystals incubated at room temperature during the study. FIG. 26 shows the distribution of drug mass in particles of various aerosolized particle diameter cutoff ranges. P2 stabilized nanocrystals prepared without and with oleic acid were aerosolized. Both formulations had MMADs near the respirable particle size cutoff near 5.3 um. FIG. 27 shows showing DLS particle size of P2 stabilized nanocrystals before and after nebulization, as well as after incubation at room temperature during the nebulization process.

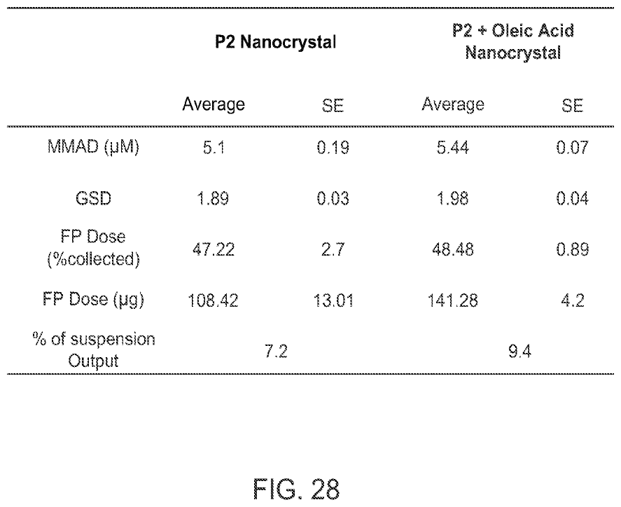

In both formulations, the FP Dose % (fraction of mass that is in respirable particles) is near 50%. The FP Dose in ug was 108.42 and 141.28 ug for the formulations prepared without and with oleic acid, respectively. The output rate is typically about 0.5 mL*min⁻¹ for these nebulizers indicating that the amount delivered was about 7.2% and 9.4% of the dose in the fine particles (respirable) using formulations prepared without and with oleic acid, respectively. See FIG. 28. These percentages compare the delivered dose in the fine particle fraction when compared to the nominal 1.5 mg*mL⁻¹ Remdesivir solution delivered over 2 minutes. Using the Next Generation Impactor (NGI) is not the most accurate measurement of total drug output, since there may be losses in sample recovery at each stage, so these measurements of drug output should be considered approximations which are likely lower than the true value.

### Inventive Examples 3-11

Block copolymers employed in Inventive Examples 3-11 are found in FIG. 29 and FIG. 30. Block copolymers used will preferably demonstrate mild foaming as described in the following experiment. Polymer in water solutions of 1% w/w were prepared at a volume of 2 mL in a 7 mL vial with diameter of 1 cm. The solution was shaken vigorously for 15 seconds and placed on the bench at room temperature. The height of the foam above the solution was measured and the time taken for the foam to reduce in height by 50% (Foam Half-Life) was observed as well. Solutions with lower foam height and lower half-life are considered to have lower foaming properties. For example, a foam height less than 3 cm and a foam half-life less than 3 hours are considered to be lower foaming properties. Foaming properties of select block copolymers used are found in the Table in FIG. 31.

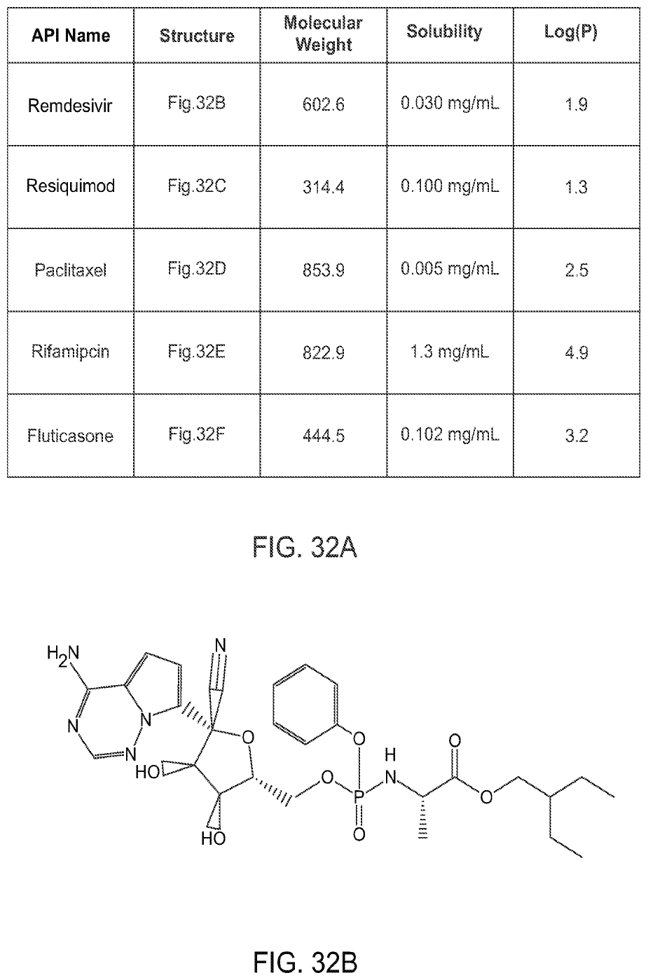

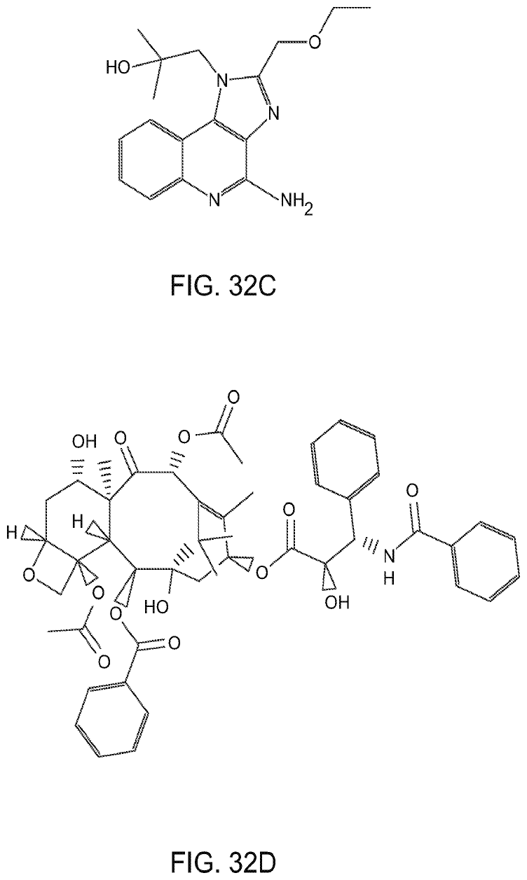

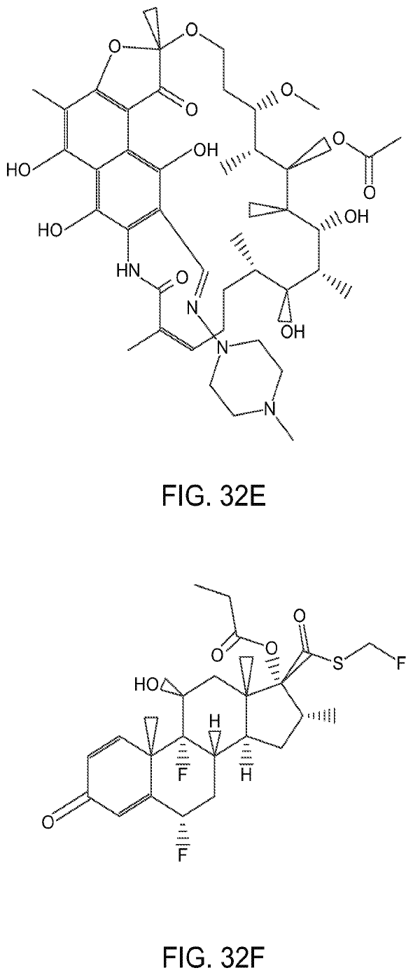

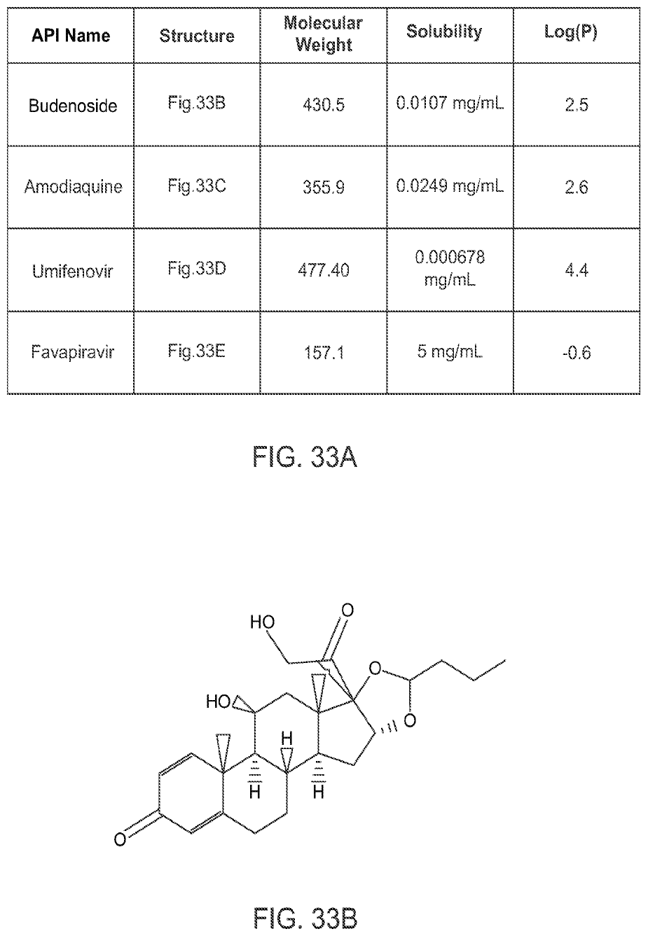

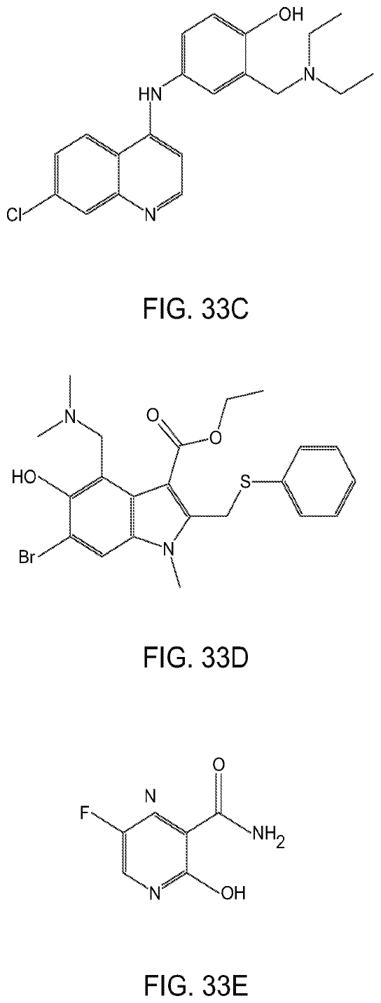

Organic compounds (active pharmaceutical ingredients/APIs) used in Examples 3-11 are found in FIG. 32 and FIG. 33.

### Examples 3A-3N—Remdesivir

In this example, API (organic compound) and Polymer stabilizer (block copolymer) are co-solubilized in the same organic solvent (Methanol) with drug stock solution ranging from 20 to 80 mg/mL (dependent on API) and polymer stock solution of 10 mg/mL. (In the case of the PBenzOx, we report size values using Ethanol as an excipient.) The API (organic compound) and Polymer Stabilizer (block copolymer) are added together to 400 μL of UltraPure Water and mixed vigorously by pipette at a final concentration of 2.5 mg/mL API (can be done at any concentration up to 20 mg/mL API). Samples were then run on DLS to determine hydrodynamic size and monitored for sample stability and precipitation. Polymer Stabilizer (block copolymer) and API (organic compound) were mixed at three different Polymer (block copolymer):API (organic compound) mass ratios: 1:100 (1%), 5:100 (5%), and 10:100 (10%). Preparation of Nanocrystals is done at room temperature (approximately 23° C.) and approximately 5 minutes passes prior to DLS measurement. Nanocrystals are then monitored for precipitation over the next 1-2 hours. Any observed precipitation or instability is noted in examples provided. Space left “blank” were not directly evaluated.

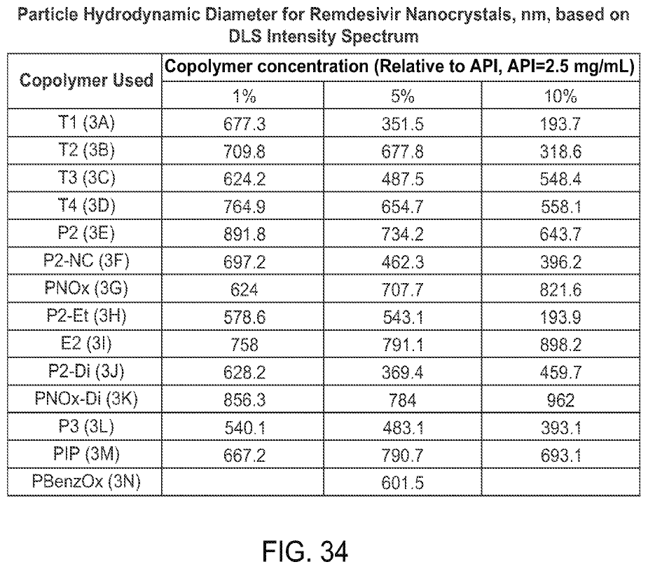

The drug Remdesivir (API or Organic compound) at 80 mg/mL is used with Polymers T1, T2, T3, T4, P2, P2-NC, PNOx, P2-Et, E2, P2-Di, PNOx-Di, P3, PBenxOx, and PIP polymers. In this embodiment, all polymers prepared stable drug nanocrystals ranging in size from 193 to 962 nm (DLS intensity distribution Hydrodynamic Diameter). See Table in FIG. 34.

### Examples 4A-E—Resiquimod

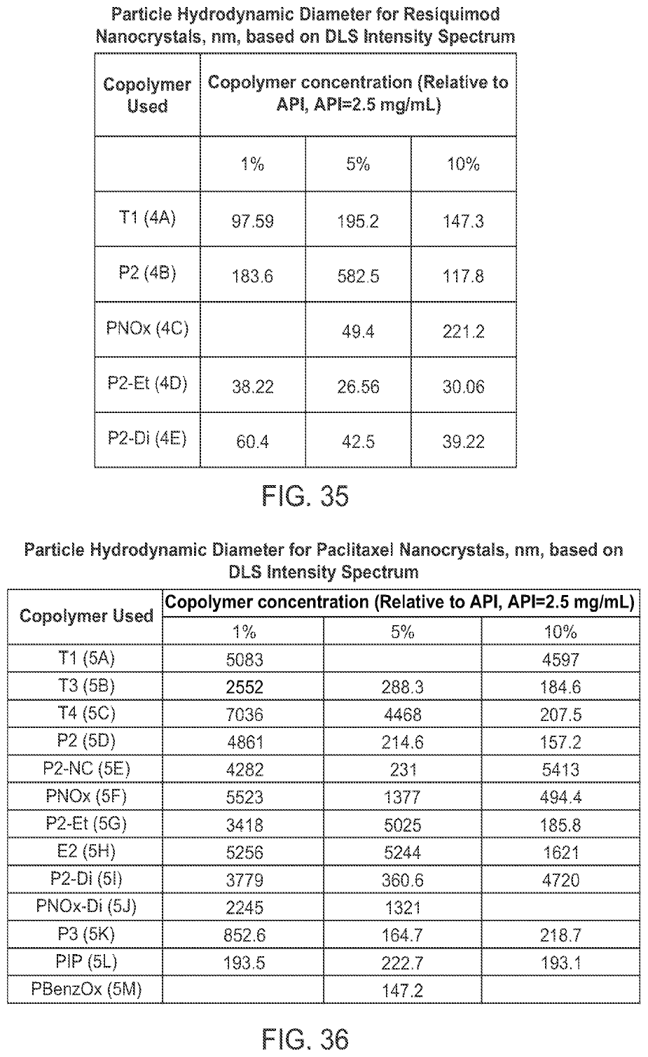

Samples were prepared the same as in Examples 3A-N utilizing the polymers T1, P2, PNOx, P2-Et, and P2-Di (block copolymers) and a Resiquimod API stock concentration of 20 mg/mL (organic compound). In this embodiment, stable drug nanocrystals were prepared ranging in size from 26.56 to 582 nm (DLS intensity distribution Hydrodynamic Diameter). See Table in FIG. 35.

### Examples 5A-M—Paclitaxel

Samples were prepared the same as Examples 3A-N utilizing the polymers T1, T3, T4, P2, P2-NC, PNOx, P2-Et, E2, P2-Di, PNOx-Di, P3, PBenzOx, and PIP (block co-polymers) with the API Paclitaxel at a 20 mg/mL API stock concentration. In this embodiment, stable drug nanocrystals ranging in size from 147.2 to 1377 nm (DLS intensity distribution Hydrodynamic Diameter) were prepared using copolymers of PBenzOx and PNOx, respectively. See Table in FIG. 36. (In the case of the PBenzOx, we report size values using Ethanol as an excipient.) Samples prepared with polymer T1, T4 (1:100 and 5:100), P2-NC (1:100), PNOx (1:100), P2-Et (1:100 and 5:100), E2, P2-Di (1:100), and PNOx-Di were unable to produce stable crystals.

### Examples 6A-C—Rifampicin

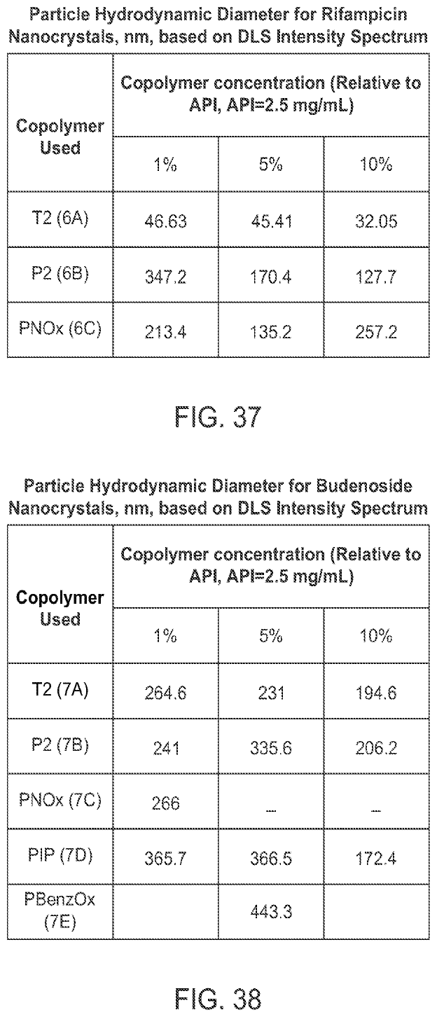

Samples were prepared the same as in Examples 3A-N utilizing the polymers T2, P2, and PNOx with a Rifampicin API stock concentration of 20 mg/mL. In this embodiment, stable drug nanocrystals were prepared ranging in size from 32.05 to 347.2 nm (DLS intensity distribution Hydrodynamic Diameter). See Table in FIG. 37.

### Examples 7A-E—Budenoside

Samples were prepared the same as in Examples 3A-N except utilizing the polymers T2, P2, PNOx, PBenzOx, and PIP (block copolymers) with a Budenoside API stock concentration of 40 mg/mL (organic compound). (In the case of the PBenzOx, we report size values using Ethanol as an excipient.) In this embodiment, stable drug nanocrystals were prepared ranging in size from 98.5 to 335.6 nm (DLS intensity distribution Hydrodynamic Diameter). See Table in FIG. 38.

### Examples 8A-C—Fluticasone

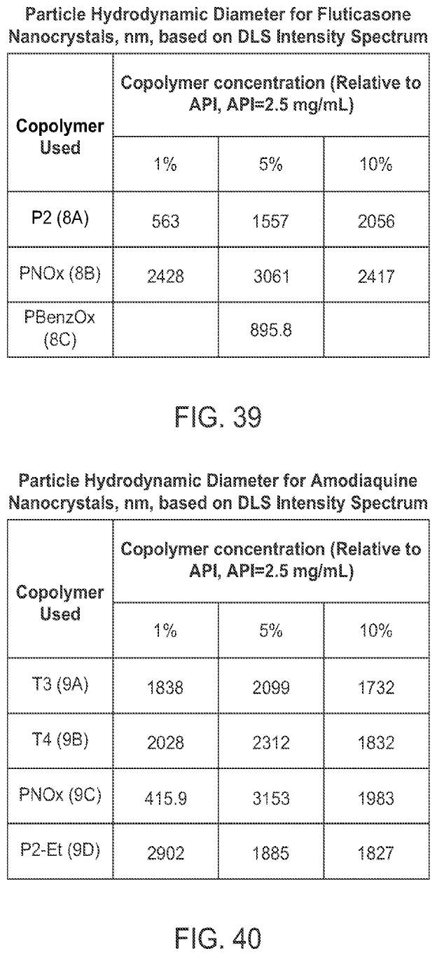

Samples were prepared the same as in Examples 3A-N, except utilizing the polymers P2, PBenzOx, and PNOx with a Fluticasone API stock concentration of 20 mg/mL. In this embodiment, stable drug nanocrystals were prepared at 563 nm and 89.8 nm (DLS intensity distribution Hydrodynamic Diameter), using the P2 and PBenzOx polymers, respectively. (In the case of the PBenzOx, we report size values using Ethanol as an excipient.) See Table in FIG. 39. P2 polymer (10:100) and PNOx were unable to produce stable nanocrystals.

### Examples 9A-D—Amodiaquine

Samples were prepared the same as in Examples 3A-N, except utilizing the polymers T3, T4. PNOx, and P2-Et with a Amodiaquine API stock concentration of 20 mg/mL. In this embodiment, stable drug nanocrystals were prepared at 415.9 nm (DLS intensity distribution Hydrodynamic Diameter). See Table in FIG. 40. Only PNOx (1:100) was able to produce stable nanocrystals.

### Examples 10A-E—Umifenovir

Samples were prepared the same as in Examples 3A-N except utilizing the polymers T1, T2, P2, PNOx, and P2-Et with a Umifenovir API stock concentration of 20 mg/mL. In this embodiment, stable drug nanocrystals were prepared at sizes ranging from 24.67 to 522.1 nm (DLS intensity distribution Hydrodynamic Diameter). See Table in FIG. 41.

### Examples 11A-C—Favapiravir

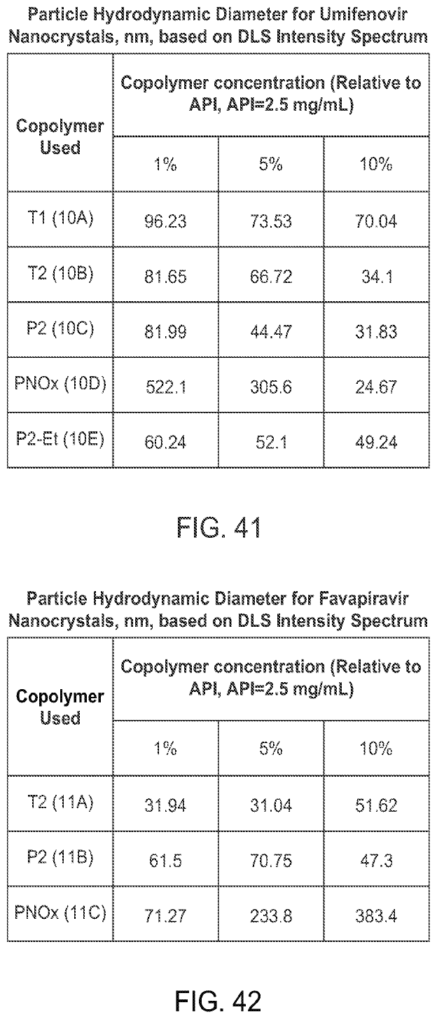

Samples were prepared the same as in Examples 3A-N, except utilizing the polymers T2, P2, and PNOx with a Favapiravir API stock concentration of 20 mg/mL. In this embodiment, stable drug nanocrystals were prepared at sizes ranging from 31.04 to 383.4 nm (DLS intensity distribution Hydrodynamic Diameter). See Table in FIG. 42.

### Example 12

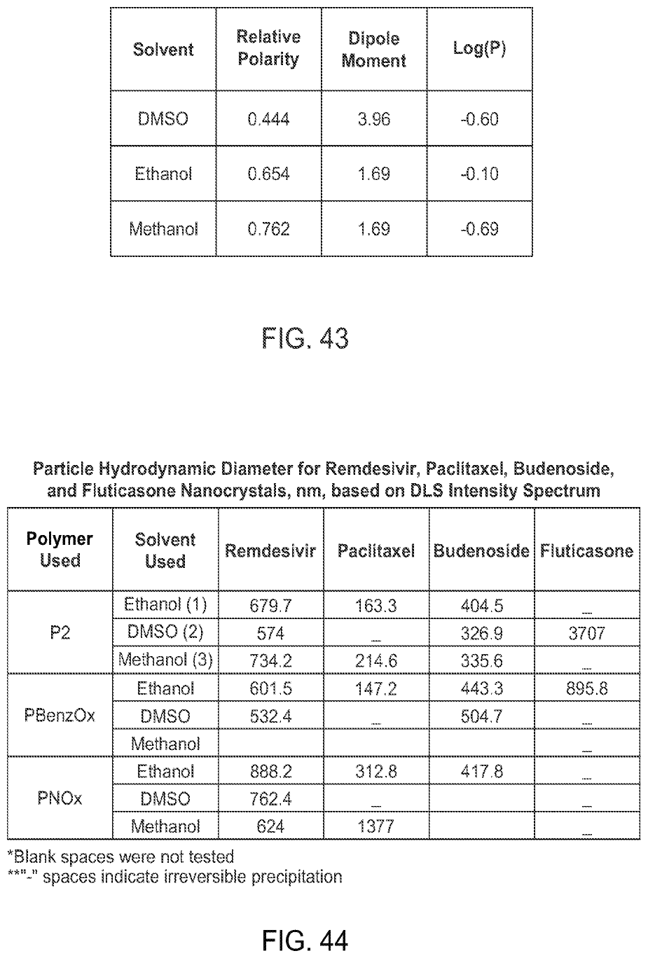

In this example, the experiment was performed exactly as described in like in Examples 3A-N (Remdesivir), 5A-M (Paclitaxel), 7A-E (Budenoside), and 8A-C (Fluticasone) above, but utilizing additional solvents as alternatives to methanol. In particular, ethanol and DMSO are used as alternative solvents. Properties of these solvents are found in the Table in FIG. 43. Additional solvents not used here could be, but are not limited to, acetonitrile, chloroform, and DMF. In this experiment, we utilized only the 5:100 polymer:API w/w ratio. Three different co-polymers (P2, PBenzOx, and PNOx) were used. For example, P2 was combined with Remdesivir like in Example 3, except that ethanol and DMSO were also tested as solvents. Resulting particles diameter using these different solvents based on DLS intensity spectrum is found in FIG. 44.

### Example 13—Nanocrystal Preparation Using Wet Milling Process

Wet milling is a “top-down” attrition-based process, in which drug is dispersed first in an aqueous based surfactant/stabilizer/polymer (excipients) solution. The resulting suspension is subjected to wet milling using a pearl/ball milling apparatus—NETZSCH MicroCer Horizontal Bead Mill (NETZCH Fine Particle Technology, LLC). Yittria-stabilized Zirconia beads is used as grinding media. An excellent fracture toughness and smooth surface of these beads allows for elimination of product contamination from both media and mill parts. Initial studies employ wet-milling method in a small volume batch (75 mL) to determine the bead size, milling duration, temperature, and polymer excipients necessary to obtain particles with desired size ranges. Briefly, each of the API in the table is supplemented with 25 ml solution of a block copolymer. The block copolymers are T1, T2, T3, T4, P2, P2-NC, PNOx, P2-Et, E2, P2-Di, PNOx-Di, and P3. The block copolymer: API weight:weight ratio is varied as follows: 0.1:100, 1:100, 5:100, 10:100, or 20:100. The beads are added to achieve a bead to suspension ratio of approx. 2 for effective milling. The beads are primary media for particle size reduction. Therefore, the YSZ beads of different diameters (in the range from 0.1 to 0.8 mm) are used and the optimal bead size will be determined. Suspensions are milled for 15 min, 30 min, 1 h, 2 h, and 6 h. In select cases, the milling duration is increased to up to 24 hours. Once the optimal milling conditions are established the milling is scaled-up to a volume of 500 ml using the same NETZSCH apparatus to produce nanosuspensions for further testing. The particle sizes, the width of particle size distribution and the zeta-potentials will be determined at different points during the milling process by dynamic light scattering (DLS) and nanoparticle tracking (NTA). Prior to the measurement, the samples are diluted with the corresponding buffer to a suitable scattering intensity and redispersed by handshaking. The net surface charge of the particles formed in nanosuspensions is monitored by measuring particle's zeta-potential.

### Methods and Materials

Materials: Methyl Triflate, 2-Methyl-2-oxazoline, 2-n-Butyl-2-oxazoline, Acetonitrile, Ether, Acetone, Deuterated Water, 3.5 kDa Dialysis membrane, Potassium carbonate, Methanol, Tyloxapol, Oleic Acid, Piperidine, Ultra Pure Water, phosphate-buffered saline (PBS), and 3.5 kDa Slidealyzers were purchased from Sigma Aldrich. Remdesivir and Tyloxapol were purchased from Medkoo, Pluronic F127 (F127) was obtained from BASF. Sulfobutylether-β-cyclodextrin (SBECD) was purchased from Medchem Express. A Salter Labs 8900 jet nebulizer (SunMed; Grand Rapids, MI) was used for all aerosol studies. Impaction stages were washed with methanol (Fisher Scientific).

Polymer Synthesis: Triblock copolymer P[MeOx-b-BuOx-b-MeOx], P2 with degrees of polymerization of the blocks 38-33-38 was synthesized according to previously described methods. For the synthesis of polymers, methyl tri-fluoromethanesulfonate (MeOTf), MeOx, and BuOx were dried by refluxing over calcium hydride (CaH₂) under inert nitrogen gas and subsequently distilled. Briefly, MeOTf was added to a reaction flask under low oxygen and water vapor conditions and in argon gas environment (1 eq.). 3 mL of dry acetonitrile was used as a solvent. MeOx was added at desired molar ratio (38 eq.) and mixed overnight at 80° C. Complete monomer consumption and block length were confirmed by ¹H-NMR (methanol solvent). BuOx was added to the reaction mixture under dry conditions at the desired block length (33 eq.) and mixed overnight at 80° C. ¹H-NMR was again used to confirm completion and block length. MeOx was then added and the reaction mixture was stirred overnight at 80° C. (38 eq.). Terminating Piperidine was added in 3-fold molar excess and the reaction mixture was mixed overnight at room temperature. Potassium Carbonate was added to dry the mixture. The mixture was then gravity filtered and washed with acetone. Acetone was removed by Rotovap, and then the mixture was added to ice cold ether in a 9:1 ether:reaction mixture volumetric ratio. The vials were then centrifuged at 1000×G for 5 minutes to pellet the precipitated polymer. Ether was decanted and the precipitate was dissolved in ultrapure water and dialyzed (3.5 kDa membrane) against water for 4 days changing the surrounding medium every 24 hours. Samples were lyophilized and final structure confirmed by ¹H-NMR.

Bottom-Up Nanocrystal Synthesis: Remdesivir nanocrystals were prepared by the solvent-anti solvent precipitation method. Briefly, remdesivir was solubilized in methanol at a concentration of 80 mg*mL⁻¹ and polymers (F127, P2, Tyloxapol) were dissolved to a concentration of 10 mg*mL⁻¹ in methanol. Oleic acid was used at a working concentration of 1 mg*mL⁻¹ in methanol. Nanocrystal suspensions were synthesized at Remdesivir concentrations ranging from 2.5-20 mg*mL⁻¹. The desired volume of suspension in UltraPure Water (Ranging from 2-10 mL in this work) was placed in a scintillation vial under rapid stirring (900 rpm). Remdesivir and polymer stabilizers were mixed together in the desired ratios with a given weight % of stabilizer as determined by Equation 1. For example, in a 2 mL, 5 mg*mL⁻¹ suspension, 125 μL of Remdesivir and 50 μL of P2 would be used. These were added to the scintillation vial under rapid stirring and allowed to mix for 30 seconds. The suspensions were then transferred to microcentrifuge tubes and centrifuged at 10000×G for 5 minutes to pellet most of the suspension. Supernatant was removed and the nanocrystals could then be redispersed to the desired concentration. For long term storage, samples were frozen in liquid nitrogen with a concentration of 20% trehalose (v/v) as a cryopreservant.

$$Mass Stabilizer / Mass Remdesivir * 100 \qquad Equation 1$$

SBECD Formulation Preparation: Remdesivir ICs were prepared by adding remdesivir in methanol dropwise at roughly 7 drops/minute to an aqueous solution of SBECD under vigorous stirring at a 1:30 Remdesivir:SBECD ratio by mass creating a roughly 2 mg*mL⁻¹ remdesivir solution. After drops finished, the solution was stirred for an additional 1 hour. The solution was then put on rotoevaporator to remove methanol and then lyophilized to form a powder which is 3.2% remdesivir by weight. Solutions were hydrated to the desired concentration in ultra-pure water and allowed to stabilize for 15 minutes before use.

Nanocrystal Size and Zeta Potential Characterization: Nanocrystal particle sizes were measured by DLS where both the intensity distribution and the number-based distribution measurements were used to gain insights about the formulation. Morphology was determined using Transmission Electron Microscopy (TEM). Zeta Potential of nanocrystals were measured using a Particle Metrix Zetaview Instrument at 20-fold dilution in 0.1×PBS.

In Vitro Drug Release Studies: Remdesivir nanocrystals and SBECD IC's were prepared at 2 mg*mL⁻¹ and 5 mg*mL⁻¹ and the release rate from the formulations was measured under sink conditions in PBS at 37° C. 100 μL of sample was placed in a 3.5 kDa slidealyzer dialysis devices after washing the membrane with PBS. Samples were incubated in triplicate at time points of 0.5, 1, 2, 4, 8, and 24 hours. The solution was removed from the dialysis device and lyophilized. It was then rehydrated in 100 μL of methanol and drug concentration was measured by HPLC after centrifugation and removal of salts.

HPLC Analysis of Remdesivir: Remdesivir concentrations were analyzed by Agilent 1100 HPLC system on a Reverse Phase C18 column from Supelco. Briefly, 70 μL of remdesivir to be measured in methanol was added to 30 μL of HPLC water for sample analysis. Mobile phase was 70:30 methanol:water ratio with a 1.0 mL*min⁻¹ flow rate. Column temperature was set to 40° C. and the retention time of remdesivir was about 5 minutes. Injection volume was 10 μL. Remdesivir was analyzed at 280 nm on a UV Photodiode Array detector.

Methanol Detection by NMR: For quantifying methanol removal from the nanocrystal suspensions, ¹H-NMR was used. ¹H-NMR spectra were recorded on an INOVA 400 at room temperature. The spectra were calibrated using the solvent signals (D₂O 4.80 ppm). Samples of nanocrystal suspensions were dissolved in deuterated water before and after centrifugations. Samples were centrifuged to pellet remdesivir nanocrystals and then supernatant was analyzed on ¹H-NMR. The methanol solvent peak area near 3.3 ppm was used to determine the amount of methanol remaining in the nanocrystal suspension.

In Vitro Antiviral Studies: Antiviral studies were performed according to previous protocols. The A549 cells expressing Ace2 (A549-Ace2) were seeded at 1×10⁴ cells/well in black walled clear bottom 96 well plates the day prior to treatment and infection. Drug stocks and controls were prepared fresh within an hour of each replicate and diluted in saline prior to addition to the 96 well plates. Prepared drug stocks were then diluted 1:100 in cell maintenance media (Gibco DMEM supplemented with 10% heat-inactivated FBS, 1% Gibco NEAA, 1% Gibco Pen-Strep) to achieve a 2× concentration. Maintenance media was removed from cells and cells were pre-treated for 1 hr with 2× drug. Cells were then either mock infected or infected at a Multiplicity of Infection (MOI)=0.02 with SARS-CoV-2-nLuc. The final concentration of drug after the addition of inoculum was 1×. Cells were then incubated for 48 hr. at 37° C. with 5% CO₂. Virus growth was measured with NanoGlo Luciferase Assay System (Promega) and cell viability was measured with CellTiter-Glo 2.0 Cell Viability Assay (Promega). Luminescence was measured using a Promega GloMax Explorer System plate reader. Background luminescence from vehicle treated wells subtracted from all other treated wells. Drug treatment experiments were performed in three independent experiments in technical duplicate.

Aerosol Generation Inertial Impaction and Aerodynamic Characterization: Inertial impaction was performed using a Next Generation Impactor (NGI; MSP Corp.; MN, USA) following United States Pharmacopeia (USP) method <1601>. Impactor stages were precooled to 4° C. for at least 90 minutes before experiments. Vacuum for the NGI was set to 15 L*min⁻¹ and a solenoid wired to the vacuum pump was set to 2 minutes. Aliquots of the remdesivir formulation were synthesized and frozen at 10 mg*mL⁻¹, thawed and then diluted to 1.5 mg*mL⁻¹. The nebulizer (Nebutech 8960) was filled with 3 mL of the remdesivir formulation and affixed to the inlet of the NGI via custom mouthpiece adapter. The nebulizer was actuated by connecting the pressurized air tubing (50 psi) and generating the aerosol at an airflow rate of 8 L*min⁻¹. Next the solenoid was switched on and nebulized material was collected over the 2 minutes. This was repeated thrice with fresh NGI plates, inlets, and Remdesivir nanocrystal suspensions.

The impactor size segregates the aerosol with cut-off aerodynamic diameters of A, B, C, D, E, F, G μm for stages 1-7, respectively, from which the particle size distribution can be constructed (USP<1601>). NGI Impactor stage cutoff diameters at the specified conditions are as follows: Stage 1=>14.1 um, Stage 2=<14.1 um, Stage 3=<8.61, Stage 4=<5.39 um, Stage 5=<3.3 um, Stage 6=<2.08 um, Stage 7=<1.36 um, and Stage 8=<0.98 um. The deposited aerosol on the inlet, stages 1-7, and multi-orifice filter (MOC) of the NGI was assayed by HPLC as described above to quantify remdesivir mass. The values were used to construct an aerodynamic particle size distribution (APSD) from which MMAD, geometric standard deviation (GSD) (Equation 2), and fine particle dose (FP Dose; mass particles<5.39 μm at 15 L*min⁻¹) could be determined. MMAD was calculated by plotting the cumulative mass percent undersize of the deposited remdesivir against its corresponding stage effective cutoff diameter, translating to a probability (PROBIT) vs. logarithm effective cutoff diameter plot, and fitting a line through points on either side of 50^{th} percentile mass collected and solving for particle size (equal to 0 on PROBIT scale). GSD was similarly calculated by determining the particle size at the 84^{th} and 16^{th} percentile (1 and −1, respectively, on PROBIT scale) and taking the square root of the former divided by the latter. The FP Dose was determined by summing the mass remdesivir collected on stages 4 through MOC of the NGI. The FP Dose % was determined by the percentage of delivered remdesivir which was in the respirable fractions. The percent of the suspension output is the mass of remdesivir delivered divided by the nominal dose.

$$GSD = [ \% mass of distribution at {D}_{84} / \% mass of distribution at {D}_{16} ] \qquad Equation 2$$

¹H-NMR of Polymers: ¹H-NMR spectra were recorded on an INOVA 400 at room temperature. The spectra were calibrated using the solvent signals (MeOD 3.31 ppm).

1. A composite particle comprising:
a nanocrystal of an organic compound; and
block copolymer associated with one or more surfaces of the nanocrystal of an organic compound, the block copolymer comprising at least one hydrophilic poly(2-oxazoline) block.

2. The composite particle of claim 1, having an average size of 70 to 1,000 nm.

3. The composite particle of claim 1, wherein a ratio of the block copolymer to the organic compound ranges from 1:100 to 10:100 by weight.

4. The composite particle of claim 1, wherein the organic compound is present in an amount of at least 80 weight percent of the particle.

5. The composite particle of claim 1, wherein the organic compound is present in an amount of at least 90 weight percent of the particle.

6. The composite particle of claim 1, wherein the organic compound has a solubility<10 mg/mL in water in a pH range from 4 and 10 at 20° C.

7. The composite particle of claim 1, wherein the organic compound has a Log(P) in a range from −2.0 to 10.0.

8. The composite particle of claim 1, wherein the organic compound is a pharmaceutical agent, a therapeutic agent, or combinations thereof.

9. The composite particle of claim 1, wherein the organic compound comprises one or more selected from the group consisting of Remdesivir, Resiquimod, Paclitaxel, Rifampicin, Fluticasone, Budenoside, Amodiaquine, Umifenovir, and Favapiravir.

10. The composite particle of claim 1, wherein the block copolymer exhibits at least one of a foam height (cm) of less than 3 cm and a foam half-life (hr.) of less than 3 hr.

11. The composite particle of claim 1, wherein the block copolymer further comprises a second block that is more hydrophobic than the hydrophilic poly(2-oxazoline) block.

12. The composite particle of claim 1, wherein the hydrophilic poly(2-oxazoline) block comprises poly(2-methyl-2-oxazoline) and/or poly(2-ethyl-2-oxazoline).

13. The composite particle of claim 11, wherein the second block comprises a more hydrophobic poly(2-oxazoline) and/or poly(2-oxazine).

14. The composite particle of claim 11, wherein the second block includes monomer having a side chain comprising a heteroaryl moiety.

15. The composite particle of claim 14, wherein the heteroaryl moiety is a five-membered ring or six membered ring.

16. The composite particle of claim 11, wherein the heteroaryl moiety is of the formula: 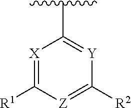
wherein X, Y and Z are heteroatoms independently selected from the group consisting of N, O, and S, and R¹ and R² are independently selected from the group consisting of hydrogen, alkyl, cycloalkyl, alkenyl, aryl, heteroaryl, amine, —SH, hydroxyl, halo, carboxyl, and —CONR³, wherein R³ is alkyl and [symbol] represents a point of attachment of the heteroaryl moiety to the monomer.

17. The composite particle of claim 1, wherein the hydrophilic poly(2-oxazoline) block is of the formula: 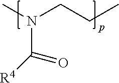
wherein R⁴ is selected from the group consisting of alkyl, cycloalkyl and alkenyl, each optionally substituted with hydroxyl, —SH, —COOR⁵, —NR⁶₂, —CONR⁷ or —CHO, wherein R⁵-R⁷ are independently selected from the group consisting of hydrogen and alkyl and wherein p is at least 5.

18. A lyophilized powder comprising:
composite particles, the composite particles including a nanocrystal of an organic compound, and block copolymer associated with one or more surfaces of the nanocrystal of an organic compound, the block copolymer comprising at least one hydrophilic poly(2-oxazoline) block.

19. The lyophilized powder of claim 18, wherein the block copolymer further comprises a second block that is more hydrophobic than the hydrophilic poly(2-oxazoline) block.

20. The lyophilized powder of claim 18, wherein the composite particles have an average size of 100-1000 nm.

21. The lyophilized powder of claim 18, wherein the organic compound is present in an amount of at least 80 weight percent of the lyophilized powder.

22. (canceled)

23. A pharmaceutical composition comprising:
composite particles including a nanocrystal of an organic compound, and block copolymer associated with one or more surfaces of the nanocrystal of an organic compound, the block copolymer comprising at least one hydrophilic poly(2-oxazoline) block.

24. The pharmaceutical composition of claim 23, further comprising one or more pharmaceutically acceptable excipients.

25. The pharmaceutical composition of claim 23, wherein the pharmaceutical composition is a dispersion comprising:
an aqueous or aqueous-based continuous phase; and
a dispersed phase comprising the composite particles.

26. The pharmaceutical composition of claim 25, wherein the composite particles have a polydispersity index (PDI) of less than 0.3.

27-40. (canceled)
